SEQUENCE LISTING (10 sequences)

SEQ ID NO: 1 - protein, 278 aa, synthetic construct
TKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 2 - protein, 248 aa, synthetic construct
AAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 3 - protein, 389 aa, synthetic construct
AMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 4 - protein, 383 aa, synthetic construct
LELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 5 - protein, 245 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSSRSSSSGGGGSGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 6 - DNA, 834 bp, synthetic construct
  source 1..834: mol_type = other DNA
acgaagaacgagaagaagtccctcaaccagagcctggccgagtggaagctcttcatctacaacccgaccaccggagaattcctggggcgcaccgccaagagctggggtttgatcttgctcttctacctagttttttatgggttcctggctgcactcttctcattcacgatgtgggttatgcttcagactctcaacgatgaggttccaaaataccgtgaccagattcctagcccaggactcatggtttttccaaaaccagtgaccgcattggaatatacattcagtaggtctgatccaacttcgtatgcagggtacattgaagaccttaagaagtttctaaaaccatatactttagaagaacagaagaacctcacagtctgtcctgatggagcactttttgaacagaagggtccagtttatgttgcatgtcagtttcctatttcattacttcaagcatgcagtggtatgaatgatcctgattttggctattctcaaggaaacccttgtattcttgtgaaaatgaacagaataattggattaaagcctgaaggagtgccaaggatagattgtgtttcaaagaatgaagatataccaaatgtagcagtttatcctcataatggaatgatagacttaaaatatttcccatattatgggaaaaaactgcatgttgggtatctacagccattggttgctgttcaggtcagctttgctcctaacaacactgggaaagaagtaacagttgagtgcaagattgatggatcagccaacctaaaaagtcaggatgatcgtgacaagtttttgggacgagttatgttcaaaatcacagcacgtgca

SEQ ID NO: 7 - DNA, 744 bp, synthetic construct
  source 1..744: mol_type = other DNA
gctgccggcacagtcttcactaccgtagaagaccttggctccaagatactcctcacctgctccttgaatgacagcgccacagaggtcacagggcaccgctggctgaaggggggcgtggtgctgaaggaggacgcgctgcccggccagaaaacggagttcaaggtggactccgacgaccagtggggagagtactcctgcgtcttcctccccgagcccatgggcacggccaacatccagctccacgggcctcccagagtgaaggctgtgaagtcgtcagaacacatcaacgagggggagacggccatgctggtctgcaagtcagagtccgtgccacctgtcactgactgggcctggtacaagatcactgactctgaggacaaggccctcatgaacggctccgagagcaggttcttcgtgagttcctcgcagggccggtcagagctacacattgagaacctgaacatggaggccgaccccggccagtaccggtgcaacggcaccagctccaagggctccgaccaggccatcatcacgctccgcgtgcgcagccacctggccgccctctggcccttcctgggcatcgtggctgaggtgctggtgctggtcaccatcatcttcatctacgagaagcgccggaagcccgaggacgtcctggatgatgacgacgccggctctgcacccctgaagagcagcgggcagcaccagaatgacaaaggcaagaacgtccgccagaggaactcttcc

SEQ ID NO: 8 - DNA, 1167 bp, synthetic construct
  source 1..1167: mol_type = other DNA
gcaatgtttatggtgaaaaatggcaacgggaccgcgtgcataatggccaacttctctgctgccttctcagtgaactacgacaccaagagtggccctaagaacatgacctttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcatgctgatccccatcgctgtgggtggtgccctggcggggctggtcctcatcgtcctcatcgcctacctcgtcggcaggaagaggagtcacgcaggctaccagactatc

SEQ ID NO: 9 - DNA, 1149 bp, synthetic construct
  source 1..1149: mol_type = other DNA
ttggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtg

SEQ ID NO: 10 - DNA, 735 bp, synthetic construct
  source 1..735: mol_type = other DNA
gaagtgcagctggtggaaagcggcggcggcctggtgcagcccgggggaagcctgcggctgagttgcgccgcttctggcttcaacatcaaagatacctacatccactgggtgagacaggcccctggcaaaggcctggagtgggtggccagaatctaccccacaaatggctacaccaggtatgccgatagcgtcaagggcagatttaccatcagcgccgacacttcaaagaacaccgcctatctgcagatgaactccctgcgcgccgaagacaccgccgtgtactactgttctcggtggggcggcgatggcttctatgccatggactactggggccagggcacactggtgaccgtgtctagcgggggaagctccaggtccagctcttccggcggcggcggctccggcggaggaggcgacatccagatgacccagtcaccaagttctctgagcgcttccgtgggcgatcgcgtgaccatcacctgccgggcttcacaggacgtgaacaccgccgtggcctggtaccagcagaaacctggcaaagcccccaaactcctgatctactccgccagtttcctgtattctggcgttcctagtagatttagtggaagcaggagcgggactgatttcactctgaccattagctctctgcagcctgaggactttgccacatattattgccagcagcactacaccacccccccaactttcggccagggcaccaaggtcgagatcaaa

(19) United States
(12) Patent Application Publication
(10) Pub. No.: US 2026/0007610 A1
(43) Pub. Date: Jan. 8, 2026

(54) CELL-DERIVED VESICLES ENGINEERED WITH ANCHOR PROTEINS AND USE THEREOF

(71) Applicant: MDIMUNE INC., Seoul (KR)

(72) Inventors: Yerim KWON, Seoul (KR); Sung-Soo PARK, Soul (KR); Seung Wook OH, Seoul (KR); Shin Gyu BAE, Seoul (KR)

(73) Assignee: MDIMUNE INC., Seoul (KR)

(21) Appl. No.: 18/992,410

(22) PCT Filed: Jul. 13, 2023
(86) PCT No.: PCT/KR2023/010049

§ 371 Date: Jan. 8, 2025

(30) Foreign Application Priority Data
Jul. 13, 2022 (KR) 10-2022-0086418
Jul. 12, 2023 (KR) 10-2023-0090542

(51) Int. Cl. A61K 9/50; A61K 47/68; A61K 47/69; A61P 35/00

(52) U.S. Cl. CPC A61K 9/5068; A61K 47/6855; A61K 47/6901; A61P 35/00

(57) ABSTRACT

The present invention relates to engineered cell-derived vesicles (CDVs) that can be used as a drug delivery system, and was completed by discovering four types of anchor proteins that match the intrinsic characteristics of CDVs and can mediate the stable introduction of biologically active molecules. The anchor proteins are CDV-specific membrane proteins that are abundantly present, and it was confirmed that CDVs comprising the anchor proteins can be more stably loaded with biologically active molecules. For example, as a result of carrying out comparative experiments by using a fluorescent protein, it was confirmed that CDVs into which the anchor proteins are introduced were more effectively loaded with the fluorescent protein, compared to CDVs without the anchor proteins. It was also confirmed that, when a cancer cell-targeting antibody was loaded into the engineered CDVs of the present invention, the engineered CDVs exhibited an increased ability to target cancer cells and were more effectively absorbed into cancer cells. That is, the CDVs of the present disclosure are BioDrone engineered with the anchor proteins and can be stably loaded with various biologically active molecules and deliver same to a target of interest, and thus are expected to be used as a platform for the delivery of various drugs and treatment.

### TECHNICAL FIELD

The present disclosure relates to cell-derived vesicles engineered with anchor proteins, a use for drug delivery thereof, and the like.

The present disclosure claims the benefit of Korean Patent Application No. 10-2022-0086418, filed on Jul. 13, 2022, and Korean Patent Application No. 10-2023-0090542, filed on Jul. 12, 2023, the entire contents of which are incorporated herein by reference.

### BACKGROUND ART

Most drugs diffuse through the body to cause side effects in cells or tissues without lesions. To minimize these side effects, active targeting research using a drug delivery system (DDS) is being actively conducted. The drug delivery system is a medical technology that delivers drugs only to a target site while protecting an off-target site by suppressing unnecessary distribution of drugs. Various drug delivery technologies are utilized to enhance therapeutic effects at a desired site of action while minimizing side effects by controlling the absorption, distribution, and excretion of nucleic acids, proteins, or other small molecules. Since effective drug delivery systems may reduce the cost and time required to develop new drugs, the United States, Japan, and the like have concentrated on the development of drug delivery systems as well as new drugs since the 1980s.

To date, liposomes, viruses, recombinant proteins, cationic polymers, and various nanomaterials have been used as carriers for drug delivery. However, cationic polymers and liposomes based on the cationic polymers have too high cytotoxicity to have an unsuitable problem for clinical application. Alternatively, methods have been attempted to chemically modify nucleic acid molecules so that nucleic acids may directly pass through the cell membrane stably, but such methods consume a lot of time and money, and require complex processes to be unsuitable for clinical applications. In addition, various nanoparticle-based drug delivery systems, such as graphene quantum dots, magnetic particles, and metal nanoparticles, have been developed, but these particles have disadvantages of high cytotoxicity, a structure that is unfavorable for the intracellular introduction of biopolymers such as nucleic acid molecules, and low intracellular delivery efficiency. Therefore, there is a continuing need for the development of drug delivery systems that not only effectively deliver various biological materials, including nucleic acids, into cells, but are also biocompatible and have a low risk of side effects.

Extracellular vesicles are nano-scale membrane structures that mediate intercellular communication by delivering cellular materials such as proteins between cells. Extracellular vesicles (EVs) that are biocompatible and have intercellular signaling functions are attracting attention as a DDS, and the EVs may not only preserve the active materials inherent in the cell membrane and cytoplasm, but also contain new materials, thereby exhibiting various biological activities. In particular, if a protein with target tissue-specific binding ability is expressed on the surface of the EV, the targeting ability of a target site in the body may be maximized, and thus it is expected to be a next-generation drug delivery system.

However, natural vesicles are secreted from cells in extremely small quantities and require considerable effort to be collected and concentrated, which has a limitation in commercialization. Therefore, artificial cell-derived vesicles (CDVs) that may be easily mass-produced through a mass extrusion method are utilized. The CDVs are distinguished from other vesicles such as exosomes or extracellular vesicles, and are utilized as analogues of natural extracellular vesicles such as exosomes and ectosomes in various research and industrial fields. Thereafter, impurities may be separated and purified from crude CDVs to easily obtain a large amount of CDVs. Since the membrane proteins and lipids of the CDV are derived from the plasma membranes or organelle membranes of a parent cell of the CDV, the membrane or membrane inside includes physiologically active molecules (proteins, lipids, sugars, etc.), that have been expressed by the cell. In addition, the CDVs have an advantage of maximizing therapeutic efficacy because the CDVs may additionally encapsulate or bind various pharmaceuticals with different properties in addition to the functions of an active material derived from the cell. In order to develop the CDVs into a DDS, a technology capable of stably and efficiently introducing targeting ligands and therapeutic drugs is required.

### DISCLOSURE

### Technical Problem

The present disclosure has been derived as a result of extensive research to solve the above-mentioned problems, and completed by discovering anchor membrane proteins (also referred to as anchor proteins) that match the intrinsic characteristics of cell-derived vesicles (CDVs) and may mediate stable loading of biologically active molecules. The CDV according to the present disclosure is an engineered CDV in which the anchor proteins are overexpressed, and various molecules such as therapeutic drugs or targeting ligands may be stably loaded through the overexpressed anchor proteins, and the molecules may be effectively delivered to a target of interest.

Accordingly, an object of the present disclosure is to provide cell-derived vesicles in which anchor proteins are overexpressed.

Another object of the present disclosure is to provide a cell (or cell line) for producing the cell-derived vesicle.

Yet another object of the present disclosure is to provide a method for producing the cell-derived vesicles.

Still another object of the present disclosure is to provide a drug delivery composition including the cell-derived vesicle as an active ingredient.

However, technical objects to be achieved in the present disclosure are not limited to the aforementioned objects, and other technical objects not described above will be apparently understood to those skilled in the art from the following disclosure of the present disclosure.

### Technical Solution

An aspect of the present disclosure provides a cell-derived vesicle in which an anchor protein is overexpressed, in which the anchor protein is at least one selected from the group consisting of Basigin, ATP1B3, LAMP1, and LAMP2.

In an embodiment of the present disclosure, the anchor protein may be inserted into the membrane of the cell-derived vesicle, but is not limited thereto.

In another embodiment of the present disclosure, the cell-derived vesicle may be derived from a cell in which the anchor protein is overexpressed, but is not limited thereto.

In yet another embodiment of the present disclosure, the cell-derived vesicle may be obtained by extruding the cell, but is not limited thereto.

In yet another embodiment of the present disclosure, the cell may be at least one selected from the group consisting of stem cells, immune cells, blood cells, embryonic cells, adipocytes, and embryonic kidney cells, but is not limited thereto.

In yet another embodiment of the present disclosure, the anchor protein may be present at a higher level in the cell-derived vesicle than a cell from which the cell-derived vesicle is derived or an exosome produced from the cell, but is not limited thereto.

In yet another embodiment of the present disclosure, the anchor protein may or not be glycosylated, but is not limited thereto.

In yet another embodiment of the present disclosure, the anchor protein may bind to the biologically active molecule, but is not limited thereto.

In yet another embodiment of the present disclosure, the biologically active molecule may be located outside or inside the membrane of the cell-derived vesicle, but is not limited thereto.

In yet another embodiment of the present disclosure, the biologically active molecule may be at least one selected from the group consisting of peptides, proteins, glycoproteins, nucleic acids, carbohydrates, lipids, glycolipids, compounds, natural products, viruses, semi-synthetic drugs, quantum dots, fluorochromes, and toxins, but is not limited thereto.

In yet another embodiment of the present disclosure, the protein may be at least one selected from the group consisting of an antibody, an antibody fragment, a growth factor, an enzyme, a nuclease, a transcription factor, an antigenic peptide, a hormone, a transport protein, an immunoglobulin, a structural protein, a motor protein, a signaling protein, a linker protein, a viral protein, a natural protein, a recombinant protein, a protein complex, a fluorescent protein, a therapeutic protein, a chemically modified protein, and prions, but is not limited thereto.

In yet another embodiment of the present disclosure, the antibody may be at least one selected from the group consisting of a full-length antibody, Fab, Fab′, F(ab′)₂, scFv, (scFv)₂, scFv-Fc, a minibody, a diabody, and a nanobody, but is not limited thereto.

In yet another embodiment of the present disclosure, the biologically active molecule may be a targeting ligand, and the cell-derived vesicle may bind to a cell expressing a target of the targeting ligand, but is not limited thereto.

Another aspect of the present disclosure provides a cell for producing the cell-derived vesicle.

Another aspect of the present disclosure provides a method for producing a cell-derived vesicle including: (S1) introducing a recombinant vector containing an anchor protein-coding gene into a cell; and
(S2) extruding the cell into which the recombinant vector has been introduced to obtain a cell-derived vesicle,
in which the anchor protein is at least one selected from the group consisting of Basigin, ATP1B3, LAMP1, and LAMP2.

In an embodiment of the present disclosure, the introduction of the recombinant vector into the cell may be performed by at least one selected from the group consisting of lentivirus, retrovirus, adenovirus, adeno-associated virus, herpes simplex virus, and vaccinia virus including the recombinant vector, but is not limited thereto.

In another embodiment of the present disclosure, the cell may be infected with a virus of 1 to 30 multiplicity of infection (MOI), but is not limited thereto.

In yet another embodiment of the present disclosure, the recombinant vector may further include a biologically active molecule-coding gene, and the biologically active molecule may be expressed in a state of binding to the anchor protein, but is not limited thereto. That is, the biologically active molecule and the anchor protein may be expressed in the form of a fusion protein.

Yet another aspect of the present disclosure provides a cell for producing a cell-derived vesicle, in which an exogenous anchor protein-coding gene may be introduced, in which the anchor protein may be at least one selected from the group consisting of Basigin, ATP1B3, LAMP1, and LAMP2.

Yet another aspect of the present disclosure provides a cell-derived vesicle obtained by extruding the cell.

Yet another aspect of the present disclosure provides a drug delivery composition (or drug delivery system) including a cell-derived vesicle in which an anchor protein is overexpressed as an active ingredient, in which the anchor protein may be at least one selected from the group consisting of Basigin, ATP1B3, LAMP1, and LAMP2.

Yet another aspect of the present disclosure provides a use of a cell-derived vesicle overexpressing the anchor protein for drug delivery.

Yet another aspect of the present disclosure provides a use of a cell-derived vesicle overexpressing the anchor protein for producing a drug delivery composition (or drug delivery system).

Yet another aspect of the present disclosure provides a method for drug delivery to a subject, cell, tissue, and/or organ, including administering a cell-derived vesicle in which an anchor proteins is overexpressed to the subject, cell, tissue, and/or organ in need thereof.

Yet another aspect of the present disclosure provides a pharmaceutical composition for the prevention or treatment of disease, including a cell-derived vesicle in which an anchor protein is overexpressed as an active ingredient, in which the cell-derived vesicle is loaded with a drug for the prevention or treatment of disease, and the anchor protein is at least one selected from the group consisting of Basigin, ATP1B3, LAMP1, and LAMP2. In an embodiment of the present disclosure, the disease may be cancer and the drug may be an anticancer agent. In another embodiment of the present disclosure, the disease may be a brain disease, and the drug may be a brain disease therapeutic agent.

Yet another aspect of the present disclosure provides a use of a cell-derived vesicle in which an anchor protein is overexpressed for the prevention or treatment of disease, in which the cell-derived vesicle is loaded with a drug for the prevention or treatment of the disease.

Yet another aspect of the present disclosure provides a use of a cell-derived vesicle overexpressing the anchor protein for preparation of a drug for the prevention or treatment of a disease.

Yet another aspect of the present disclosure provides a method for preventing or treating disease, including administering a cell-derived vesicle in which an anchor protein is overexpressed to a subject in need thereof, in which the cell-derived vesicle is loaded with a drug for preventing or treating the disease.

In an embodiment of the present disclosure, the drug may be at least one selected from the group consisting of an antibody or fragment thereof, a therapeutic protein, and a therapeutic peptide, but is not limited thereto.

In another embodiment of the present disclosure, the drug may be bound to the anchor protein of the cell-derived vesicle; or loaded into the inside or membrane of the cell-derived vesicle, but is not limited thereto.

In yet another embodiment of the present disclosure, the cell-derived vesicle may further include a targeting ligand, and the targeting ligand may be bound to the anchor protein and positioned outside the membrane of the cell-derived vesicle, but is not limited thereto.

In yet another embodiment of the present disclosure, the cell-derived vesicle may bind to a cell expressing a target of the targeting ligand, but is not limited thereto.

In yet another embodiment of the present disclosure, the anticancer agent may be bound to an anchor protein, but is not limited thereto.

### Advantageous Effects

The present disclosure relates to engineered cell-derived vesicles (CDVs) that can be used as a drug delivery system, and was completed by discovering four types of anchor proteins that match the intrinsic characteristics of CDVs and can mediate the stable introduction of biologically active molecules. The anchor proteins are CDV-specific membrane proteins that are abundantly present, and it was confirmed that CDVs comprising the anchor proteins can be more stably loaded with biologically active molecules. For example, as a result of carrying out comparative experiments by using a fluorescent protein, it was confirmed that CDVs into which the anchor proteins are introduced were more effectively loaded with the fluorescent protein, compared to CDVs without the anchor proteins. It was also confirmed that, when a cancer cell-targeting antibody was loaded into the engineered CDVs of the present disclosure, the engineered CDVs exhibited an increased ability to target cancer cells and were more effectively absorbed into cancer cells. That is, the CDVs of the present disclosure are BioDrone engineered with the anchor proteins and can be stably loaded with various biologically active molecules and deliver same to a target of interest, and thus are expected to be used as a platform for the delivery of various drugs and treatment.

### DESCRIPTION OF DRAWINGS

FIG. 1A is a table summarizing candidate anchor proteins that are identified as being abundantly present in cell-derived vesicles (CDVs) compared to cells or exosomes.

FIG. 1B is a genetic schematic diagram of a lentiviral vector for expression of candidate anchor proteins.

FIGS. 2A and 2B show results of flow cytometry to confirm the transfection efficiency (FIG. 2A) and the expression level of an EGFP gene included in a vector (FIG. 2B) after transfecting HEK293 cells with a vector for expressing candidate anchor proteins.

FIG. 3 shows results of comparing the transduction efficiency by detecting GFP-positive cells, GFP fluorescence intensity, and the like by flow cytometry under various conditions, after treating adherent HEK293 cells (top) or suspension HEK293 cells (bottom) with virus particles expressing candidate anchor proteins GFP, PTGFRN, and RAB7A at different MOIs to determine optimal transduction conditions.

FIGS. 4A and 4B show results of confirming the transduction efficiency after transducting cells with lentivirus under an optimal condition (MOI5) to construct cell lines expressing candidate anchor proteins. FIG. 4A shows results of detecting GFP fluorescence 24 hours after transduction, and FIG. 4B shows results of evaluating the transduction efficiency (left graph) and GFP intensity (right graph) by flow cytometry.

FIG. 5 shows results of identifying GFP expressing cells using flow cytometry to select cells expressing fusion proteins at high levels.

FIGS. 6A to 6D show results of confirming overexpression of respective fusion proteins (candidate anchor proteins) in a cell line constructed using lentivirus. Transduction efficiency (FIG. 6A) and GFP intensity (FIG. 6B) were confirmed by flow cytometry after cell sorting, and GFP was quantified using GFP ELISA (FIG. 6C). The expression of candidate anchor proteins was evaluated by Western blot using antibodies against each protein (FIG. 6D).

FIGS. 7A to 7C show results of confirming candidate anchor proteins present in CDVs after obtaining cell-derived vesicles (anchor-CDVs) from cells expressing candidate anchor proteins. FIG. 7A shows a histogram obtained from nanoparticle flow cytometer measurement results of GFP-positive CDVs, and FIGS. 7B and 7C show GFP signal intensity and quantification results of CDVs expressing anchor-GFP fusion proteins, respectively.

FIGS. 8A and 8B are diagrams showing CDVs including anchor proteins selected for a BioDrone platform. FIG. 8A is a schematic diagram showing anchor proteins selected from HEK2593 cells and GFP fluorescence thereof. FIG. 8B shows results of evaluating anchor proteins using a nanoparticle flow cytometer and ELISA. The number of GFP(+) particles and GFP proteins per CDV is shown in Table.

FIG. 9 is a flowchart (top) showing a process of extruding anchor-CDVs from cells using a membrane filter or a depth filter, and a table (bottom) summarizing CDV extrusion conditions of two anchor protein-overexpressing cells HEK-BSG and HEK-LAMP1.

FIGS. 10A to 10C show results of comparing the characteristics of BioDrone anchor-CDVs according to an extrusion method. FIG. 10A shows a histogram of GFP(+) particles, FIG. 10B shows the number and GFP intensity of GFP(+) particles in anchor-CDVs extruded through a membrane filter or depth filter, and FIG. 10C shows results of detecting anchor proteins using Western blot.

FIG. 11 shows results of topology analysis of anchor proteins through protease cleavage analysis. BioDrone anchor-CDVs were treated with various concentrations of Proteinase K (PK), and each tag protein was detected through Western blot. A 3× Flag tag at the N-terminus and a HA tag at the C-terminus of the anchor protein were detected using HRP-conjugated anti-Flag or anti-HA, respectively.

FIG. 12A is a gene cleavage map of a recombinant vector expressing anchor protein-trastuzumab scFv (scTTZ) fusion proteins.

FIG. 12B is a diagram showing structures of LAMP1-scTTZ fusion proteins, as representative examples of anchor protein-scTTZ fusion proteins. For detection of the fusion proteins, miRFPnano3 and nanoLuciferase were added to the C-terminus.

FIGS. 13AA to 13C show results of confirming the expression of anchor protein-scTTZ fusion proteins after producing transduced cell lines expressing the fusion proteins using lentivirus. FIG. 13A (FIGS. 13AA and 13AB) shows results of flow cytometry of miRFPnano3 fluorescence in cells 24 hours after transduction. Three weeks after puromycin selection, miRFPnano3 fluorescence intensity (FIG. 13B) and luciferase activity (FIG. 13C) were measured to confirm that the expression rates of the fusion proteins were 90% or higher.

FIG. 14 shows results of Western blot analysis of scTTZ fusion proteins expressed in Expi293F. The fusion proteins were detected using antibodies targeting respective anchor proteins BSG, ATP1B3, LAMP1, and LAMP2, anti-Flag antibodies, and protein L.

FIGS. 15A and 15B show results of confirming whether anchor protein-trastuzumab scFv (scTTZ) fusion proteins bind to a target HER2. FIG. 15A is a drawing showing the binding of the fusion protein and a FITC-labeled HER2 recombinant protein (HER2-FITC). FIG. 15B shows results of detecting HER2-FITC binding to anchor protein-scTTZ expressing cells using flow cytometry and determining a relative mean fluorescence intensity (MFI) of FITC.

FIG. 16 shows results of evaluating ratios of RFP+ vesicles among total CDV particles to confirm whether ssTTZ is stably introduced into CDV.

FIGS. 17A and 17B show results of analyzing the characteristics of LAMP1-CDV introduced with scTTZ (FIG. 17A, Western blot results; FIG. 17B, results of analyzing ratios of vesicles bound to HER2-FITC compared to RFP+ vesicles).

FIG. 18 shows results of flow cytometry of HER2-FITC binding of scTTZ-gLAMP1-CDV and scTTZ-LAMP1-CDV to determine an effect of glycosylation of an anchor protein LAMP1.

FIG. 19 shows results of confirming the HER2 expression level by cancer cell.

FIG. 20 shows results of measuring the degree of cell binding of CDV by flow cytometry after treating gLAMP1-CDV with or without scTTZ introduction into CT26 (HER2-negative cell line) or CT26/hHER2 (human HER2 gene-expressing cell line) to determine the binding ability of anchor-CDV introduced with scTTZ to HER2-expressing cells.

FIGS. 21A to 21C show results of comparing the degree of cell binding of CDV after treatment with gLAMP1-CDV with or without scTTZ introduction in HER2 high-expressing cell lines BT-474, SK-BR-3 and an HER2-negative cell line (MDA-MB-231) (FIG. 21A, a histogram of fluorescence peak shift due to CFSE-labeled CDV binding; FIG. 21B, quantitative results of CDV binding for each cell line; FIG. 21C, degree of CDV binding confirmed through fluorescence measurement in HER2 high-expressing cell lines).

FIGS. 22A and 22B show results of confirming the uptake of anchor-CDV introduced with scTTZ into HER2-expressing cells over time (FIG. 22A) and results of confirming the fold change of scTTZ-gLAMP1-CDV compared to gLAMP1-CDV uptake into cells (FIG. 22B).

FIG. 23 shows results of evaluating ratios of RFP+ vesicles among total CDV particles to confirm whether cetuximab is stably introduced into CDV.

FIG. 24 shows results of comparing rates of GFP introduction into CDV through fluorescence signal analysis after introducing GFP, a fluorescent protein, into CDV with or without using the anchor proteins of the present disclosure.

### BEST MODE

The present disclosure relates to engineered cell-derived vesicles (CDVs) that may be used as a drug delivery system, and was completed by discovering four types of anchor proteins that match the intrinsic characteristics of CDVs and may mediate the stable introduction of biologically active molecules. The anchor proteins are CDV-specific membrane proteins that are abundantly present, and it was confirmed that CDVs including the anchor proteins may be more stably loaded with biologically active molecules compared to general CDVs.

Specifically, in an embodiment of the present disclosure, 12 types of candidate anchor membrane proteins that are abundantly present in CDVs compared to cells or exosomes were selected through proteomic analysis of CDVs, cell lines overexpressing these candidate anchor proteins were constructed, and the cell lines were extruded to obtain CDVs expressing the anchor proteins. By comparing the expression level, distribution, and the like of the anchor protein in each CDV, four types of proteins BSG, ATP1B3, LAMP1, and LAMP2 that are most effectively introduced into CDVs were selected. It was confirmed that the selected anchor proteins maintain the topologies in CDVs as in cells and maintain the characteristics regardless of an extrusion method of CDVs. Accordingly, the four types of proteins were selected as anchor proteins for engineering the CDVs of the present disclosure as confirmed to be useful for CDV modification, such as fusing targeting ligands or introducing therapeutic cargos as membrane proteins that are stably present on CDV particles (Example 1).

Accordingly, it was confirmed whether CDVs expressing the selected anchor proteins may actually be utilized as a DioDrone for delivery of biologically active molecules. An scFv site of an antibody trastuzumab for HER2 protein was used as a targeting ligand, and a cell line overexpressing a fusion protein of the targeting ligand and an anchor protein was produced and extruded to produce a HER2-targeting CDV (HER2-anchor CDV). As CDV analysis results, it was confirmed that the HER2 targeting scFv was stably introduced into the CDV, and it was confirmed that the CDV effectively targets and binds to HER2-expressing cancer cells through the scFv loaded on the surface (Example 2).

In addition, as a result of preparing a CDV loaded with GFP, a fluorescent protein, it was confirmed that the CDV into which the anchor protein of the present disclosure was introduced loaded GFP more effectively than the CDV into which the anchor protein was not introduced, and thus it was confirmed that the active ingredient or cargo may be loaded into the CDV with higher efficiency by using the anchor protein of the present disclosure (Example 3).

As such, the CDVs of the present disclosure are BioDrones engineered with the anchor proteins and may be stably loaded with various biologically active molecules and deliver the various biologically active molecules to a target of interest, and thus are expected to be used as a platform for the delivery of various drugs and treatment.

Hereinafter, the present disclosure will be described in detail.

A main object of the present disclosure is to provide cell-derived vesicles (CDVs) in which anchor proteins are overexpressed.

In the present disclosure, the “cell derived vesicles (CDVs)” refer to vesicles artificially produced from nucleated cells. The CDVs may be produced by being released from the cell membrane of almost all types of cells and may have a double phospholipid membrane structure, which is the structure of the cell membrane. The cell-derived vesicles of the present disclosure are distinguished from vesicles naturally secreted from cells, such as exosomes. As used throughout this specification, the term “vesicles” refer to vesicles in which an inside and an exterior are separated by a lipid bilayer composed of cell membrane components of a derived cell, cell membrane lipids, cell membrane proteins, nucleic acids, cell components, and the like of the cell are included, and the size is smaller than an original cell, but are not limited thereto.

The cell-derived vesicles according to the present disclosure have micrometer-level sizes. For example, the diameter of the CDV may be less than 0.2 μm. More specifically, the diameter of the CDV may be 10 to 100 nm, 20 to 100 nm, 50 to 100 nm, 50 to 300 nm, 50 to 400 nm, 50 to 500 nm, 50 to 250 nm, 50 to 200 nm, 50 to 180 nm, 50 to 160 nm, 50 to 150 nm, 50 to 100 nm, 100 to 300 nm, 100 to 250 nm, 100 to 200 nm, 100 to 180 nm, 120 to 300 nm, 150 to 300 nm, or 130 to 170 nm, but is not limited thereto.

The CDV according to the present disclosure may have sizes of nanometers to micrometers. For example, the diameter of the CDV may be 50 to 500 nm, 50 to 400 nm, 50 to 300 nm, 50 to 200 nm, 50 to 150 nm, 100 to 500 nm, 100 to 400 nm, 100 to 300 nm, 100 to 200 nm, 100 to 150 nm, 130 to 500 nm, 130 to 400 nm, 130 to 300 nm, or 130 to 200 nm, but is not limited thereto.

In addition, the CDV according to the present disclosure may have a positive surface charge. For example, the zeta potential of the CDV may be −20 to +50 mV, −20 to +30 mV, −20 to +20 mV, −20 to +10 mV, −20 to +0 mV, −20 to −5 mV, −20 to −10 mV, −15 to −5 mV, −15 to −10 mV, −13 to −10 mV, −12 to −10 mV, +10 mV to +50 mV, +20 mV to +50 mV, +30 mV to +50 mV, or +40 mV to +50 mV, but is not limited thereto.

The CDV according to the present disclosure may be produced by using a method selected from the group consists of extrusion, sonication, lysis, homogenization, freeze-thawing, electroporation, chemical treatment, mechanical degradation, and treatment with a physical stimulus applied externally to the cells for a suspension containing nucleated cells, but is not limited thereto. For example, the CDV of the present disclosure may be obtained by a method of extruding the suspension containing cells using an extruder. The extrusion force of the extruder applied to produce the CDV may be 5 to 200 psi, 10 to 150 psi, 10 to 100 psi, 10 to 50 psi, 10 to 40 psi, or 50 to 100 psi.

In addition, the cell-derived vesicles may first perform removing cell nuclei before extruding a sample containing the cells into micropores. The cell nuclei may be removed by centrifugation.

In addition, the vesicles according to the present disclosure may be obtained by extruding the sample containing cells into micropores, and preferably, the vesicles may be obtained by sequentially extruding cells using micropores having a larger size to a smaller size. The diameter of the micropores may be 0.01 to 100 μm, 0.01 to 80 μm, 0.01 to 60 μm, 0.01 to 40 μm, 0.01 to 20 μm, 0.01 to 15 μm, 0.01 to 10 μm, 0.01 to 7 μm, 0.01 to 3 μm, 0.01 to 1 μm, 0.01 to 0.5 μm, 0.01 to 0.1 μm, 0.1 to 20 μm, or 0.1 to 0.5 am, but is not limited thereto. For example, the sequential extrusion may be performed sequentially using a filter having a pore diameter of 5 to 20 μm, a filter having a pore diameter of 2 to 7 μm, a filter having a pore diameter of 0.7 to 3 μm, and a filter having a pore diameter of 0.1 to 0.5 μm. The size of the micropores may be appropriately adjusted depending on a type of cells used. The CDV obtained through the process may be subjected to an additional purification process.

In addition, the vesicles according to the present disclosure may also be produced through a producing method including extruding a sample containing cells through a depth filter. The retention rate of the above depth filter may be 0.1 to 1 μm, 0.1 to 0.9 μm, 0.1 to 0.8 μm, 0.1 to 0.7 μm, 0.1 to 0.65 μm, 0.2 to 1 μm, 0.4 to 1 μm, 0.5 to 1 μm, 0.6 to 1 μm, 0.2 to 0.8 μm, 0.4 to 0.8 μm, 0.4 to 0.7 μm, 0.5 to 0.7 μm, or 0.6 to 0.7 μm, but is not limited thereto. The extrusion force of the depth filter may be 5 to 200 psi, 10 to 150 psi, 10 to 100 psi, 10 to 50 psi, 10 to 40 psi, or 50 to 100 psi, but is not limited thereto. In an embodiment of the present disclosure, the cell-derived vesicle of the present disclosure may be obtained by introducing a suspension containing cells into an assembly equipped with a depth filter for extrusion of cells, and then extruding the suspension by applying pressure. The pressure may be the pressure of nitrogen (N₂) gas. For example, when producing CDVs by passing cells through a depth filter, the CDVs may be extruded by applying nitrogen gas pressure of 0.1 to 5 bar, 0.1 to 4 bar, 0.1 to 3 bar, 0.1 to 2.5 bar, 0.3 to 3 bar, 0.1 to 2.5 bar, or 1 to 2.5 bar, but is not limited thereto. The CDV obtained through the process may be subjected to an additional purification process.

The purification process is intended to remove other materials (vesicles smaller than the CDVs of the present disclosure or other contaminants such as proteins, nucleic acids, etc.) and obtain only desired CDVs, and may be preferably performed by adding a buffer solution in an amount of 1 to 5 times, 1 to 4 times, or 1 to 3 times the total volume of the cell-derived vesicle and liposome mixture solution.

Specifically, the CDV produced through the extrusion step may be purified through a tangential flow filtration (TFF) process. The membrane used at this time preferably has a cut off of 800 kDa, 750 kDa, 700 kDa, 650 kDa, 600 kDa, 500 kDa, 450 kDa, 400 kDa, 350 kDa, or 300 kDa or more, but is not limited thereto. In the TFF process, a concentration step of concentrating the CDV suspension to a concentration of 5 times or more and a buffer exchange step are performed. Through the process, the CDVs are concentrated and impurities are removed.

After obtaining the CDVs through the TFF process, the method may further perform a size exclusion chromatography process for additional purification. At this time, it is preferable to use a column having a recovery range of about 35 to 350 nm or 70 to 1,000 nm, and since the CDVs corresponds to relatively large particles, the CDVs may be obtained by collecting fractions eluted through the column. Through such a purification process, impurities such as vesicles smaller than CDVs and other proteins may be removed, and high-purity, uniform nano-sized cell-derived vesicles may be obtained.

In the present disclosure, the “sample containing cells” may be a sample containing nucleated cells or transduced cells thereof, and is a concept that includes all of a culture solution, suspension, dilution, etc. of the cells. Here, the cell is a concept that includes, without limitation, any cell capable of producing vesicles.

Specifically, the “cell” according to the present disclosure may be used without limitation as long as the cell is any cell from which cell-derived vesicles may be separated, and includes all cells separated from natural biological entities. Preferably, the cells are nucleated cells. Additionally, the cells may be derived from any type of animal or plant, including human and non-human mammals. Accordingly, the type of cells from which the cell-derived vesicles according to the present disclosure may be obtained is not particularly limited, but for example, the cells may be stem cells, immune cells, blood cells, (human) embryonic cells, adipocytes, and/or embryonic kidney cells. As a more specific example, the cells according to the present disclosure may obtain the cell-derived vesicles from stem cells, renal cells, embryonic kidney cells, nucleated cells (HEK293), cancer cells, acinar cells, myoepithelial cells, erythrocytes, immune cells, monocytes, dendritic cells, natural killer cells, T cells, B cells, macrophages, endothelial cells, epithelial cells, neurons, glial cells, astrocytes, muscle cells, platelets, and the like. In addition, the cells according to the present disclosure may be various types of immune cells, tumor cells, stem cells, acinar cells, myoepithelial cells or platelets, and in an embodiment of the present disclosure, the stem cells may be at least one selected from the group consisting of mesenchymal stem cells, induced pluripotent stem cells, embryonic stem cells and salivary gland stem cells. In another embodiment of the present disclosure, the stem cells may be adipose-derived mesenchymal stem cells or umbilical cord-derived mesenchymal stem cells, but are not limited thereto.

In the present disclosure, the “anchor protein” means a membrane protein that is located on the membrane of the CDV and mediates the loading of the biologically active molecule onto the CDV The anchor protein is located on the CDV membrane and loads the biologically active molecule onto the CDV by binding to the biologically active molecule. The biologically active molecule may be bound to the anchor protein after producing the CDV expressing the anchor protein, or may be expressed in a cell in a form bound to the anchor protein (i.e., a fusion protein) and loaded onto the CDV. The anchor protein of the present disclosure is derived from a parent cell of the CDV, and may be a membrane protein of the plasma membrane of the cell, or a membrane protein derived from the membrane of an organelle such as a lysosome. The anchor protein of the present disclosure is a transmembrane protein and may have a structure including an extracellular domain, a transmembrane domain, and a cytoplasmic domain. Accordingly, the anchor protein of the present disclosure exists in a state of being inserted (or penetrated) into the membrane of the CDV, and other biologically active molecules may bind to the extracellular domain located outside the membrane. In addition, other labeling proteins (fluorescent proteins, tags, etc.) for detecting the anchor proteins may be bound to an intracellular domain located inside the membrane. In the present disclosure, the CDV including the anchor protein may be referred to as an anchor-CDV, a biodrone CDV, etc. In the present disclosure, the “CDV in which the anchor protein is overexpressed” refers to a CDV that contains an excessive amount of anchor protein through genetic engineering, and refers to an engineered CDV that contains a higher level of anchor protein compared to an original CDV naturally derived from the same parent cell. The anchor protein may be directly overexpressed in the CDV, but preferably, the CDV may also be engineered with the anchor protein by delivering the excessive amount of anchor protein to the CDV from the parent cell engineered with the anchor protein. In the present disclosure, engineering means introducing and expressing an exogenous gene or introducing an exogenous protein.

The anchor protein of the present disclosure is a CDV-specific protein. For example, the anchor proteins may be present at a particularly high level in CDVs compared to cells or other cell-derived organelles or cell-derived vesicles. Specifically, the anchor proteins may be present at a higher level in the CDVs than a parent cell from which the CDV is derived or exosomes or extracellular vesicles produced from the parent cell. In addition, the anchor protein may be a natural protein that exists naturally in the parent cell and is transferred to the CDV, or an exogenous protein that is expressed from an exogenous gene introduced into the parent cell and is transferred to the CDV In addition, the anchor proteins introduced into the CDVs may or not be glycosylated.

Preferably, the anchor proteins are one or more selected from the group consisting of Basigin (BSG), ATPase Na+/K+ transporting subunit beta 3 (ATP1B3), Lysosomal-associated membrane glycoprotein 1 (LAMP1), and Lysosomal-associated membrane glycoprotein 2 (LAMP2). The Basigin is a transmembrane glycoprotein of the plasma membrane that may bind to various ligands, including a cyclophilin protein and the like. General information about the Basigin may be found as Gene ID 682 in the NCBI genetic database. The ATP1B3 is a subclass of Na⁺/K⁺ and H⁺/K⁺ ATPases beta chain proteins, and is a membrane protein that maintains properly electrochemical distribution of Na and K ions on the boundary of the membrane, and is essential for osmotic pressure regulation, transport of organic/inorganic molecules, and electrical excitation of neurons. General information about the ATP1B3 may be identified as Gene ID 483 in NCBI. The LAMP1 and LAMP2 are glycoproteins present in the lysosome membrane and are involved in lysosome biogenesis, autophagy, cholesterol homeostasis, and the like. General information about the LAMP1 and LAMP2 may be identified as Gene ID 3916 and Gene ID 3920 in NCBI, respectively.

The anchor protein ATP1B3 according to the present disclosure may include an amino acid sequence represented by SEQ ID NO: 1 or consist of the amino acid sequence represented by SEQ ID NO: 1, but is not limited thereto. Alternatively, the ATP1B3 protein may be encoded by a nucleic acid molecule including a nucleotide sequence represented by SEQ ID NO: 6 or encoded by a nucleic acid molecule consisting of the nucleotide sequence represented by SEQ ID NO: 6, but is not limited thereto.

The anchor protein BSG according to the present disclosure may include an amino acid sequence represented by SEQ ID NO: 2 or consist of the amino acid sequence represented by SEQ ID NO: 2, but is not limited thereto. Alternatively, the BSG protein may be encoded by a nucleic acid molecule including a nucleotide sequence represented by SEQ ID NO: 7 or encoded by a nucleic acid molecule consisting of the nucleotide sequence represented by SEQ ID NO: 7, but is not limited thereto.

The anchor protein LAMP1 according to the present disclosure may include an amino acid sequence represented by SEQ ID NO: 3 or consist of the amino acid sequence represented by SEQ ID NO: 3, but is not limited thereto. Alternatively, the LAMP1 protein may be encoded by a nucleic acid molecule including a nucleotide sequence represented by SEQ ID NO: 8 or encoded by a nucleic acid molecule consisting of the nucleotide sequence represented by SEQ ID NO: 8, but is not limited thereto.

The anchor protein LAMP2 according to the present disclosure may include an amino acid sequence represented by SEQ ID NO: 4 or consist of the amino acid sequence represented by SEQ ID NO: 4, but is not limited thereto. Alternatively, the LAMP2 protein may be encoded by a nucleic acid molecule including a nucleotide sequence represented by SEQ ID NO: 9 or encoded by a nucleic acid molecule consisting of the nucleotide sequence represented by SEQ ID NO: 9, but is not limited thereto.

Meanwhile, in the present disclosure, a polypeptide (or nucleic acid molecule) represented by a specific sequence may include not only the corresponding sequence but also a biological equivalent thereof. That is, when considering a variant having biologically equivalent activity of the polypeptide (nucleic acid molecule), it is interpreted that one aspect of the polypeptide (or nucleic acid molecule) also includes a sequence showing substantial identity with the sequence described in the sequence number. Specifically, the polypeptide molecule (nucleic acid molecule) consisting of the amino acid sequence (nucleotide sequence) represented by the specific sequence number is not limited only to the corresponding amino acid sequence (nucleotide sequence), and variants of the amino acid sequence (nucleotide sequence) are included within the scope of the present disclosure. The polypeptide molecule (nucleic acid molecule) consisting of the amino acid sequence (nucleotide sequence) represented by the specific sequence number of the present disclosure is a concept including functional equivalents of the polypeptide molecule (nucleic acid molecule) constituting the amino acid sequence (nucleotide sequence), for example, variants in which a part of the amino acid sequence (nucleotide sequence) of the polypeptide molecule (nucleic acid molecule) is modified by deletion, substitution or insertion, but may perform a function functionally identical to the corresponding polypeptide (nucleic acid molecule). Specifically, the polypeptide (nucleic acid molecule) disclosed in the present disclosure may include an amino acid sequence (nucleotide sequence) having a sequence homology of at least 70%, more preferably at least 80%, even more preferably at least 90%, and most preferably at least 95% with the amino acid sequence represented by the specific sequence number. For example, the polypeptide (nucleic acid molecule) includes a polypeptide (nucleic acid molecule) having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%. The “% of sequence homology” with the polypeptide (nucleic acid molecule) is identified by comparing two optimally arranged sequences with a comparison region, and a part of the polypeptide sequence (nucleotide sequence) in the comparison region may include addition or deletion (i.e., gap) compared to a reference sequence (without addition or deletion) for an optimal alignment of the two sequences.

The anchor protein may be bound to a biologically active molecule, thereby loading the biologically active molecule onto CDV.

In the present disclosure, the “biologically active molecule” refers to a general term for materials having biological or pharmaceutical activity, and means materials (e.g., antibodies or ligands, etc.) that target a specific protein, materials that may penetrate into cells (cytoplasm or nucleus) to participate in regulating physiological activity or exhibit pharmacological effects, or materials that are transported into cells and act and have biological activity even in various parts of the body such as cells, tissues, interstitial cells, and blood. The biologically active molecule of the present disclosure may maintain intrinsic biological activity even when loaded onto CDV by binding to an anchor protein. For example, the biologically active molecules may be targeting ligands or therapeutic cargos. In the present disclosure, the targeting ligand refers to a component capable of specifically recognizing and targeting a specific material (e.g., an antigen). Preferably, the targeting ligand may target a specific antigen, etc., and induce CDV loaded with the ligand to bind to a cell expressing the specific antigen, etc. In the present disclosure, the therapeutic cargo refers to a material having a preventive, improving, and/or therapeutic effect on a disease. There is no limitation on the type of material.

In the present disclosure, the biologically active molecule is loaded onto the CDV by binding to the anchor protein, and at this time, the biologically active molecule may be located outside the membrane of the CDV (i.e., outside of the CDV), inside the membrane of the CDV (i.e., inside the lipid bilayer), or inside the CDV. That is, the biologically active molecule may be located outside or inside the membrane of the CDV, and preferably, the biologically active molecule may be located outside the membrane of the CDV by binding to the extracellular domain of the anchor protein.

In yet another embodiment of the present disclosure, the biologically active molecules may be selected from the group consisting of peptides, proteins, glycoproteins, nucleic acids, carbohydrates, lipids, glycolipids, compounds, natural products, viruses, semi-synthetic drugs, quantum dots, fluorochromes, toxins, and complexes thereof. Preferably, the biologically active molecule of the present disclosure is a protein, a glycoprotein, a peptide, or the like.

In the present disclosure, the protein may be at least one selected from the group consisting of an antibody, an antibody fragment, a growth factor, an enzyme, a nuclease, a transcription factor, an antigenic peptide, a hormone, a transport protein, an immunoglobulin, a structural protein, a motor protein, a signaling protein, a linker protein, a viral protein, a natural protein, a recombinant protein, a protein complex, a fluorescent protein, a therapeutic protein, a chemically modified protein, and prions, but is not limited thereto.

Non-limiting examples of the nucleic acid include DNA, RNA, antisense oligonucleotide (ASO), microRNA (miRNA), small interfering RNA (siRNA), aptamers, locked nucleic acid (LNA), peptide nucleic acid (PNA), morpholino, and the like.

Non-limiting examples of the compound include therapeutic drugs, toxic compounds, chemical compounds, etc. The “drug” is a broad concept that includes materials for alleviating, preventing, treating or diagnosing diseases, injury or specific symptoms. That is, the cell-penetrating peptide according to the present disclosure may be used as a drug delivery system for preventing or treating diseases.

In addition, the biologically active molecules of the present disclosure may include molecules capable of modulating the pharmacokinetics and/or pharmacodynamics of polymers, such as cholesterol, chemotherapeutics, vitamins, co-factors, 2,5-A chimeras, allozymes, aptamers, polyamines, polyamides, polyethylene glycols, and polyethers.

In an embodiment of the present disclosure, the biologically active molecule is an antibody or a fragment thereof. In the present disclosure, the “antibody” refers to an immunoglobulin molecule that immunologically has reactivity with a specific antigen (epitope), and includes all of polyclonal antibodies, monoclonal antibodies, and functional fragments thereof. In addition, the term may include forms produced by genetic engineering, such as chimeric antibodies (e.g., humanized murine antibody) and heterozygous antibodies (e.g., bispecific antibody). The antibody includes a variable region of a heavy chain and/or a light chain (VH, heavy chain variable region; VL, light chain variable region). The variable region includes a portion that forms an antigen-binding site of an antibody molecule as a primary structure, and the antibody of the present disclosure may consist of a partial fragment including the variable region. A linker may be positioned between the light chain and heavy chain variable regions of the antibody or antigen-binding fragment thereof. The “linker” refers to a polypeptide that links the light chain variable region and the heavy chain variable region to each other without damaging their intrinsic antigen-binding properties.

In the present disclosure, the antibody may be at least one selected from the group consisting of a full-length antibody, Fab, Fab′, F(ab′)₂, scFv, (scFv)₂, scFv-Fc, minibody, diabody, and nanobody, but is not limited thereto.

In the present disclosure, the term “fragment” of the antibody means a (functional) fragment having an antigen-binding function of the antibody, and preferably an antigen-binding fragment of the antibody. The fragment is used to mean not only scFv, (scFv)2, Fab, Fab′ and F(ab′)₂, but also minibodies, diabodies, nanobodies, or fragments thereof. The definitions of the fragments are well known in the art.

More specifically, the “single-chain Fv” or “scFv” antibody fragment refers to a protein in which the variable regions of the light and heavy chains of an antibody are linked to each other by a linker consisting of a peptide sequence in which about 15 amino acids are linked. These domains exist within a single polypeptide chain. An Fv polypeptide may further include a polypeptide linker between the VH and VL domains to allow the scFv to form a desired structure for antigen binding. As used in the present disclosure, the “Fv” fragment is an antibody fragment that contains intact antibody recognition and binding sites. These regions consist of a dimer of one heavy chain variable domain and one light chain variable domain, which are tightly and virtually covalently associated, for example in scFv.

The “nanobody” is an antibody variable domain consisting only of a heavy chain, also referred to as a VHH domain. The nanobody has an advantage of not only improving tissue penetration and antigen accessibility due to their small size and stable structure compared to existing antibodies, but also minimizing immune side effects due to Fc.

The antibody exhibits antigen specificity depending on changes in sequence of their variable regions. The variable region of the antigen binding site is divided into a less variable framework region (FR) and a more variable complementarity determining region (CDR), and both the heavy chain and the light chain have three CDR regions divided into CDR1, 2, and 3, and four FR regions. The complementarity determining region is a region having binding specificity to an antigen in the variable region of the antibody. The CDRs of each chain are typically referred to as CDR1, CDR2, CDR3 sequentially from an N-terminus, and are also identified by a chain in which a specific CDR is located.

The CDV according to the present disclosure may include two or more biologically active molecules. Each biologically active molecule may be the same type or of different types. For example, the CDV of the present disclosure may include a therapeutic drug together with a targeting ligand for targeting cells, tissues, or organs as a biologically active molecule. Alternatively, the CDV of the present disclosure may include a labeling protein for labeling the CDV together with a targeting ligand or therapeutic drug as a biologically active molecule. Each biologically active molecule may be bound with one anchor protein, or may be bound with different anchor proteins, respectively, or a first biologically active molecule may be bound with the anchor protein and a second biologically active molecule may be loaded onto the membrane or the inside of the CDV. When two or more types of biologically active molecules are bound with an anchor protein, the biologically active molecules may be bound sequentially with one end of the anchor protein, or may be bound with each of both ends of the anchor protein.

Those skilled in the art may select an appropriate biologically active molecule according to a purpose and load the biologically active molecule onto CDV through the anchor protein of the present disclosure. For example, the biologically active molecules may be targeting ligands that target specific tissues, organs, or cells. Therefore, CDV loaded with the targeting ligand may specifically recognize a target of the ligand. For example, when the target ligand is an antibody or a fragment thereof, the CDV loaded with the ligand may recognize and bind to a cell expressing the target (antigen) of the ligand. For example, in the case of a CDV loaded with an antibody or fragment thereof targeting brain cells or blood-brain barrier (BBB) endothelial cells, since the CDV may be moved into the brain tissue through the antibody or fragment, a brain disease treatment agent, etc. may be additionally loaded to deliver a brain-specific therapeutic agent.

In addition, the biologically active molecule may be a tumor antigen-specific antibody or fragment thereof that targets cancer cells. For example, the biologically active molecule may be a targeted anticancer agent. The tumor antigen includes not only tumor-specific antigens that are expressed only in cancer cells, but also tumor-associated antigens that may be expressed even in normal cells, but are expressed at a particularly high frequency or are more active in cancer cells. For example, the tumor antigen may be a surface protein that is expressed only in cancer cells or expressed at a higher frequency in cancer cells. As an embodiment of the present disclosure, the present inventors produced a HER2-specific CDV loaded with a fragment (scFv) of trastuzumab targeting HER2 as a biologically active molecule, and confirmed that the CDV specifically binds to HER2-expressing cancer cells.

The biologically active molecule may be linked (coupled) to the anchor protein as a complex by physical bonding, chemical bonding, covalent bonding, non-covalent bonding, peptide bonding, or self-assembly, or in an integrated or fused form using a mediator (e.g., a linker).

In an embodiment of the present disclosure, the biologically active molecule and the anchor protein may be expressed in a fused state to form a complex. For example, when a gene encoding the biologically active molecule and a gene encoding the anchor protein are inserted together into one vector and then an organism is transduced with the vector to express the genes inserted into the vector, the biologically active molecule and the anchor protein may be expressed as a fusion protein. In addition, when expressed as the fusion protein, an arbitrary linker may be included between the biologically active molecule and the anchor protein.

In addition, the present disclosure provides a cell (or cell line) for producing cell-derived vesicles according to the present disclosure. A detailed description of the cells has been described above and thus will be omitted.

Specifically, the cell may be introduced with an exogenous anchor protein-coding gene, and the anchor protein may be at least one selected from the group consisting of Basigin, ATP1B3, LAMP1, and LAMP2. That is, the cell for producing the cell-derived vesicles of the present disclosure may be a cell in which the anchor proteins are overexpressed.

The anchor protein-coding gene may be inserted into a recombinant vector and introduced into a cell. Accordingly, the present disclosure may provide a recombinant vector for producing CDVs according to the present disclosure, in which a gene encoding the anchor protein is inserted. In addition, the present disclosure may provide a cell into which the recombinant vector has been introduced. The recombinant vector may further include a biologically active molecule-coding gene together with the anchor protein-coding gene. Preferably, the biologically active molecule may be expressed in a state to be bound to the anchor protein. For example, the anchor protein-coding gene and the biologically active molecule-coding gene may be expressed in the form of a fusion protein from the recombinant vector.

In the present disclosure, “recombinant vector” refers to a vector capable of expressing a peptide or protein encoded by a heterologous nucleic acid inserted into the vector, and preferably means a vector produced to express a target protein (as used in the present disclosure, a progranulin protein or fragments thereof). The “vector” refers to any medium for the introduction and/or transition of nucleotides into a host cell in vitro, ex vivo or in vivo, and may be a replication unit (replicon) to which another DNA fragment may bind to cause replication of the bound fragment, and the “replication unit” refers to any genetic unit (e.g., plasmid, phage, cosmid, chromosome, virus, etc.) that functions as an autonomous unit of DNA replication in vivo, i.e., is capable of replicating under self-regulation.

The vector according to the present disclosure may be linear DNA, plasmid DNA or a recombinant viral vector, but is not limited thereto. The vector includes, for example, plasmid vectors, cosmid vectors, and viral vectors such as bacteriophage vectors, adenovirus vectors, lentivirus vectors, retrovirus vectors, and adeno-associated virus vectors.

The recombinant vector of the present disclosure may preferably include a promoter, which is a transcription initiation factor to which RNA polymerase binds, an arbitrary operator sequence for regulating transcription, a sequence encoding a suitable mRNA ribosome binding site and a sequence for regulating the termination of transcription and translation, a terminator, etc. More preferably, the recombinant vector may further include a polyhistidine tag (an amino acid motif consisting of at least 5 histidine residues), a signal peptide gene, an endoplasmic reticulum retention signal peptide, a cloning site, etc., and may further include a tag gene, a selection marker gene, such as an antibiotic resistance gene for selecting a transformant, etc. In the recombinant vector, the polynucleotide sequence of each of the genes is operably linked to a promoter. As used in the present disclosure, the term “operatively linked” refers to a functional linkage between a nucleotide expression regulatory sequence, such as a promoter sequence, and the other nucleotide sequence, and accordingly, the regulatory sequence regulates transcription and/or translation of the other nucleotide sequence.

In the present disclosure, the gene or the recombinant vector may be transduced or transfected into a virus producing cell, i.e., a packaging cell line. The “transfection” may be used with various types of techniques commonly used to introduce an exogenous nucleic acid (DNA or RNA) into a prokaryotic or eukaryotic host cell, such as electrophoresis, calcium phosphate precipitation, DEAE-dextran transfection, or lipofection. A virus including a target gene according to the present disclosure (a gene encoding an anchor protein or a gene encoding a fusion protein of a biologically active molecule and an anchor protein) may be proliferated within the packaging cell line and released outside the cell, and the virus may be transduced into a target cell (i.e., a cell producing the CDV of the present disclosure). The nucleic acid of the virus transduced into the cell is used to produce the target protein (anchor protein; or a fusion protein of an anchor protein and a biologically active molecule) with or without being inserted into the genome of the cell.

The present disclosure may provide an isolated cell into which a recombinant vector according to the present disclosure has been introduced (by transformation, transfection, transduction, etc.). The cell here refers to a cell for proliferating (amplifying) the recombinant vector, not a cell that ultimately produces CDV. That is, the cell represents a host cell directly transduced/transformed/transfected with the above-mentioned nucleic acid molecule or recombinant vector. For example, if the expression vector is a viral vector, the cell may be a packaging cell for producing a virus containing the viral vector. The selection of a suitable host is considered to be obvious to those skilled in the art from the suggested contents of the present disclosure.

Further, the present disclosure provides a method for producing cell-derived vesicles including: (S1) introducing a recombinant vector containing an anchor protein-coding gene into a cell; and
(S2) extruding a cell into which the recombinant vector has been introduced to obtain cell-derived vesicles.

The step (S1) is a step of introducing a recombinant vector into an appropriate host cell (e.g., transformation, transduction, transfection, etc.) so that the expression vector is replicated inside the cell or a protein and the like are expressed from the expression vector. Those skilled in the art may select an appropriate introduction method depending on a type of vector and cell. For example, if the recombinant vector is a viral vector, the vector may be introduced into the cell by infecting the cell with a virus containing the recombinant vector. For example, the recombinant vector may be introduced into a cell by lentivirus, retrovirus, adenovirus, adeno-associated virus, herpes simplex virus, and/or vaccinia virus including the recombinant vector, and preferably, the recombinant vector may be introduced into a cell by lentivirus. At this time, the cell may be infected with a virus (lentivirus, retrovirus, adenovirus, adeno-associated virus, herpes simplex virus, and/or vaccinia virus including the recombinant vector) at 1 to 30 multiplicity of infection (MOI), 1 to 20 MOI, 1 to 15 MOI, 1 to 10 MOI, 1 to 8 MOI, 1 to 7 MOI, or 1 to 5 MOI, thereby introducing the recombinant vector. When two or more types of viruses are used to introduce the recombinant vector into the cell, those skilled in the art may introduce the recombinant vector into the cell by appropriately combining the viruses within the range of 1 to 30 MOI, 1 to 20 MOI, 1 to 15 MOI, 1 to 10 MOI, 1 to 8 MOI, 1 to 7 MOI, or 1 to 5 MOI.

Accordingly, step (S1) may include culturing a cell so that a target gene introduced into the cell is expressed in the cell. The culturing may be performed for a period of time sufficient for the target protein to be expressed within the cell after the recombinant vector is introduced into the cell. More preferably, the culturing may be performed for a period of time sufficient for the target protein to be expressed in the cell and for CDV including the target protein to be produced.

Step (S2) is a step of obtaining cell-derived vesicles from a cell into which the recombinant vector is introduced to overexpress the anchor protein and/or biologically active molecule according to the present disclosure. The CDVs may be obtained by extruding a sample containing cells. The extruding may be performed using a filter or depth filter containing micropores. The process of obtaining the CDVs through cell extrusion has been described above and thus will be omitted. The CDVs obtained from step (S2) may include anchor proteins (and biologically active molecules) expressed from the recombinant vector.

The CDVs obtained through step (S2) may have a higher level of anchor proteins than the cell (parent cell) into which the recombinant vector has been introduced, or the exosome produced therefrom. Specifically, the level of the anchor protein of the CDV obtained through step (S2) may be at least 1%, 2%, 3%, 4%, 5%, 10% or higher, for example, 5%, 10%, 20%, 30%, 40%, or 50%, 60%, 70%, 80%, 90% or more higher, and/or 0.5-fold, 1.1-fold, 1.2-fold, 1.4-fold, 1.6-fold, 1.8-fold or higher, compared to the anchor protein of the parent cell or its exosome.

In addition, the present disclosure provides a drug delivery composition including cell-derived vesicles in which anchor proteins of the present disclosure are overexpressed as an active ingredient. In the present disclosure, the “delivery” means delivery into a target cell, tissue, or organ.

In the present disclosure, the drug is not limited to a specific type, and any drug (protein, peptide, lipid, carbohydrate, nucleic acid, compound, etc.) that has preventive, improving, and/or therapeutic activity for a specific disease may be included without limitation. As a non-limiting example, the drug may be at least one selected from the group consisting of an antibody or fragment thereof, a therapeutic protein, and a therapeutic peptide.

In the present disclosure, the drug is the biologically active molecule according to the present disclosure, and may be delivered to a target cell/tissue/organ in a form bound to an anchor protein. In this case, the drug may not only have pharmacological activity for preventing/improving/treating a specific disease, but may also have a targeting function of a target. For example, the drug is an antibody-based anticancer agent (anticancer antibody). That is, the CDV of the present disclosure may target cancer cells and exhibit anticancer activity through an anticancer antibody bound to an anchor protein. The anticancer antibody is not limited to a specific type, and any antibody known in the art may be included without limitation. As a non-limiting example, the anticancer antibody may be cetuximab, trastuzumab, rituximab, cixutumumab, ganitumab, dalotuzumab, figitumumab, teprotumumab, robatumumab, AVE1642, BIIB022, isiratumab, or fragments thereof.

The drug may be delivered to the target by being separately loaded into the membrane or the inside of CDV without binding to the anchor protein. In this case, the CDV may further include a targeting ligand for targeting cells/tissues/organs as a biologically active molecule bound to the anchor protein. For example, when the drug is the anticancer agent, the CDV may include an antibody or a fragment thereof that targets a tumor antigen as a targeting ligand, and may deliver the drug by specifically binding to cancer cells through the targeting ligand. Alternatively, when the drug is the brain disease therapeutic agent, the CDV may include an antibody or fragment thereof that targets brain cells and may migrate to brain tissue through the targeting ligand to deliver the drug.

In addition, the CDV of the present disclosure may have two or more biologically active molecules, and each biologically active molecule may be bound to the anchor protein. For example, the CDV of the present disclosure may include both a targeting ligand for targeting and a drug (e.g., a therapeutic protein, a therapeutic peptide, etc.), and each thereof may be bound to the anchor protein. Accordingly, the CDV may target a target through the targeting ligand and deliver a drug to the corresponding target to exhibit therapeutic activity.

The drug delivery composition may be used as a pharmaceutical composition for preventing or treating a specific disease. That is, the present disclosure provides a pharmaceutical composition for preventing or treating diseases, including cell-derived vesicles in which drugs are loaded and anchor proteins are overexpressed as an active ingredient.

For example, when the biologically active molecule according to the present disclosure has a preventive, improving, and/or therapeutic effect on a specific disease, a CDV loaded with the biologically active molecule may be used for the prevention, improvement, and/or treatment of the disease. At this time, the drug may not only have therapeutic activity, but may also have a targeting function itself. Alternatively, the CDV may further include a targeting ligand.

In another embodiment of the present disclosure, the cell-derived vesicle includes a targeting ligand as a biologically active molecule, and may further include a drug for preventing, improving, and/or treating a disease in the membrane or the inside of the CDV. The targeting ligand enables the CDV to target cells, tissues, or organs to which the drug is to be delivered for the prevention, improvement, and/or treatment of the disease.

The disease is a disease associated with the drug, and is a disease that may be prevented, treated, and/or improved by administering the drug. Accordingly, those skilled in the art may select an appropriate drug depending on a disease to be prevented, treated, and/or improved and apply the drug to the present disclosure.

For example, when the drug is an anticancer agent, the disease may be cancer. That is, the present disclosure may provide a pharmaceutical composition for preventing or treating cancer, which includes cell-derived vesicles in which the anchor proteins according to the present disclosure are overexpressed as an active ingredient, in which the cell-derived vesicle is loaded with an anticancer agent. The anticancer agent may have not only anticancer activity but also a function of targeting cancer cells (e.g., anticancer antibody). The anticancer agent may be bound to the anchor protein of the CDV membrane, or may be loaded onto the CDV in a form separated from the anchor protein (e.g., located in the membrane or the inside of the CDV). For example, the anticancer agent may be exposed outside of the CDV while bound to the anchor protein of the CDV membrane to target cancer cells, induce binding between the CDV and cancer cells, and exert anticancer activity against cancer cells. In the present disclosure, the cancer is not limited to a specific type, but the cancer may be selected from colorectal cancer, colon cancer, thyroid cancer, oral cancer, pharyngeal cancer, laryngeal cancer, cervical cancer, brain cancer, lung cancer, bladder cancer, kidney cancer, liver cancer, pancreatic cancer, prostate cancer, tongue cancer, breast cancer, uterine cancer, stomach cancer, bone cancer, lymphoma, blood cancer, squamous cell carcinoma, lung adenocarcinoma, peritoneal cancer, skin cancer, skin melanoma, ocular melanoma, rectal cancer, anal cancer, esophageal cancer, small intestine cancer, endocrine cancer, parathyroid cancer, adrenal cancer, soft tissue sarcoma, urethral cancer, gastrointestinal cancer, glioblastoma, ovarian cancer, endometrial cancer, salivary gland cancer, vulvar cancer, head and neck cancer, and the like. The anticancer agent is not limited to a specific type, and any agent known in the art having anticancer activity may be included without limitation. For example, the anticancer agent may be a protein or peptide-based anticancer agent. Alternatively, the anticancer agent may be an antibody or fragment thereof having anticancer activity. As one embodiment, the present disclosure provides a pharmaceutical composition for preventing or treating cancer, including cell-derived vesicles loaded with trastuzumab or a fragment thereof (scFv, etc.) and overexpressing anchor proteins as an active ingredient. Here, the trastuzumab or fragment thereof may be bound to the anchor proteins.

As another example, the disease may be a brain disease, and the drug loaded into the CDV may be a therapeutic agent for the brain disease. The brain disease is not limited to a specific type, but may be selected from degenerative brain disease, Parkinson's disease, Huntington's disease, Alzheimer's disease, mild cognitive impairment, senile dementia, amyotrophic lateral sclerosis, Spinocer ebellar Atrophy, Tourette's Syndrome, Friedrich's Ataxia, Machado-Joseph's disease, Lewy Body Dementia, Dystonia, Progressive Supranuclear Palsy, Frontotemporal Dementia, ischemic stroke, cerebral hemorrhage, cerebral infarction, stroke, etc. The CDV may include a targeting ligand (e.g., a brain cell- or BBB-specific antibody or fragment thereof) for targeting brain tissue.

When the composition (drug delivery composition, pharmaceutical composition, etc.) of the present disclosure is used for treating a specific disease, the content of the cell-derived vesicles for delivering the nucleic acid molecule in the composition may be appropriately controlled depending on the symptoms of a disease, the degree of progression of the symptoms, the condition of a patient, etc., and for example, may be 0.0001 to 99.9 wt %, or 0.001 to 50 wt % based on the total weight of the composition, but is not limited thereto. The content ratio is a value based on a dry amount after removing the solvent.

The composition according to the present disclosure may further include suitable carriers, excipients, and diluents which are commonly used in the preparation of the pharmaceutical composition. The excipients may be, for example, one or more selected from the group consisting of diluents, binders, disintegrants, lubricants, adsorbents, moisturizers, film-coating materials, and controlled-release additives.

The composition according to the present disclosure may be formulated and used in the form of external preparations, such as powders, granules, sustained-release granules, enteric-coated granules, liquids, eye drops, elixirs, emulsions, suspensions, alcohols, troches, aromatic waters, limonades, tablets, sustained-release tablets, enteric-coated tablets, sublingual tablets, hard capsules, soft capsules, sustained-release capsules, enteric-coated capsules, pills, tinctures, soft extracts, dry extracts, fluid extracts, injections, capsules, irrigants, sticky ointments, lotions, pastes, sprays, inhalants, patches, sterile injectable solutions, or aerosols, according to conventional methods, respectively. The external preparations may have formulations such as creams, gels, patches, sprays, ointments, sticky ointments, lotions, liniments, pastes, or cataplasmas.

The carriers, the excipients, and the diluents that may be included in the composition according to the present disclosure may include lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil.

When the composition is formulated, the formulation may be prepared by using diluents or excipients, such as a filler, an extender, a binder, a wetting agent, a disintegrating agent, and a surfactant, which are generally used.

The additives of the tablets, powders, granules, capsules, pills and troches according to the present disclosure may be used with excipients such as corn starch, potato starch, wheat starch, lactose, sucrose, glucose, fructose, D-mannitol, precipitated calcium carbonate, synthetic aluminum silicate, calcium monohydrogen phosphate, calcium sulfate, sodium chloride, sodium bicarbonate, refined lanolin, microcrystalline cellulose, dextrin, sodium alginate, methylcellulose, sodium carboxymethyl cellulose, kaolin, urea, colloidal silica gel, hydroxypropyl starch, hydroxypropyl methyl cellulose (HPMC) 1928, HPMC 2208, HPMC 2906, HPMC 2910, propylene glycol, casein, calcium lactate and Primojel; and binders such as gelatin, arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, refined shellac, starch, hydroxypropyl cellulose, hydroxypropyl methylcellulose, polyvinyl alcohol, and polyvinyl pyrrolidone. In addition, the additives may be used with disintegrants such as hydroxypropyl methylcellulose, corn starch, agar powder, methylcellulose, bentonite, hydroxypropyl starch, sodium carboxymethylcellulose, sodium alginate, carboxymethyl cellulose calcium, calcium citrate, sodium lauryl sulfate, anhydrous silicic acid, 1-hydroxypropyl cellulose, dextran, ion exchange resin, polyvinyl acetate, formaldehyde-treated casein and gelatin, alginic acid, amylose, guar gum, baking soda, polyvinyl pyrrolidone, calcium phosphate, gelled starch, arabic gum, amylopectin, pectin, sodium polyphosphate, ethyl cellulose, sucrose, magnesium aluminum silicate, di-sorbitol solution, and light anhydrous silicic acid; and lubricants, such as calcium stearate, magnesium stearate, stearic acid, hydrogenated vegetable oil, talc, lycopodium, kaolin, petrolatum, sodium stearate, cocoa butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, macrogol, synthetic aluminum silicate, anhydrous silicic acid, higher fatty acids, higher alcohols, silicone oil, paraffin oil, polyethylene glycol fatty acid ether, starch, sodium chloride, sodium acetate, sodium oleate, dl-leucine, and light anhydrous silicic acid.

Additives in the liquid formulation according to the present disclosure may be used with water, diluted hydrochloric acid, diluted sulfuric acid, sodium citrate, monostearate sucroses, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamin, polyvinylpyrrolidone, ethylcellulose, sodium carboxymethylcellulose, etc.

The syrups according to the present disclosure may be used with a solution of white sugar, other sugars or sweeteners, etc., and may also be used with a flavoring agent, a coloring agent, a preservative, a stabilizer, a suspending agent, an emulsifier, a thickener, etc., as needed.

In the emulsions according to the present disclosure, purified water may be used, and an emulsifier, a preservative, a stabilizer, a fragrance, etc. may be used as needed.

The suspensions according to the present disclosure may be used with suspending agents, such as acacia, tragacanth, methylcellulose, carboxymethylcellulose, sodium carboxymethylcellulose, microcrystalline cellulose, sodium alginate, hydroxypropylmethylcellulose (HPMC), HPMC 1828, HPMC 2906, HPMC 2910, and the like, and may be used with surfactants, preservatives, stabilizers, colorants, and fragrances as needed.

The injections according to the present disclosure may include: solvents such as distilled water for injection, 0.9% sodium chloride injection, Ringer's injection, dextrose injection, dextrose+sodium chloride injection, PEG, lactated Ringer's injection, ethanol, propylene glycol, nonvolatile oils such as sesame oil, cottonseed oil, peanut oil, soybean oil, and corn oil, ethyl oleate, isopropyl myristate, and benzene benzoate; solubilizers such as sodium benzoate, sodium salicylate, sodium acetate, urea, urethane, monoethylacetamide, butazolidine, propylene glycol, Tweens, nitrile acid amide, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃), carbon dioxide gas, sodium metabisulfite (Na₂S₂O₅), sodium sulfite (Na₂SO₃), nitrogen gas (N₂), and ethylenediaminetetraacetic acid; sulfating agents such as sodium bisulfide 0.1%, sodium formaldehyde sulfoxylate, thiourea, disodium ethylenediaminetetraacetate, and acetone sodium bisulfite; analgesics such as benzyl alcohol, chlorobutanol, procaine hydrochloride, glucose, and calcium gluconate; and suspending agents such as CMC sodium, sodium alginate, Tween 80, and aluminum monostearate.

The suppositories according to the present disclosure may be used with base materials, such as cocoa butter, lanolin, witepsol, polyethylene glycol, glycerogelatin, methylcellulose, carboxymethylcellulose, a mixture of stearic acid and oleic acid, subanal, cottonseed oil, peanut oil, palm oil, cocoa butter+cholesterol, lecithin, lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydroxocote SP, S-70-XXA, S-70-XX75 (S-70-XX95), Hydrokote 25, Hydrokote 711, Idropostal, Massa estrarium (A, AS, B, C, D, E, I, T), Massa-MF, Masupol, Masupol-15, Neosupostal-N, Paramound-B, Suposiro (OSI, OSIX, A, B, C, D, H, L), suppository base type IV (AB, B, A, BC, BBG, E, BGF, C, D, 299), Supostal (N, Es), Wecovi (W, R, S, M, Fs), and Tezester triglyceride bases (TG-95, MA, 57).

Solid formulations for oral administration include tablets, pills, powders, granules, capsules, etc., and these solid formulations are prepared by mixing the extract with at least one excipient, such as starch, calcium carbonate, sucrose or lactose, gelatin, etc. Further, lubricants such as magnesium stearate and talc may be used in addition to simple excipients.

Liquid formulations for oral administration may correspond to suspensions, oral liquids, emulsions, syrups, and the like, and may include various excipients, such as wetting agents, sweeteners, flavoring agents, preservatives, and the like, in addition to water and liquid paraffin which are commonly used as simple diluents. Formulations for parenteral administration include sterile aqueous solutions, non-aqueous solvents, suspensions, emulsions, lyophilized preparations, and suppositories. As the non-aqueous solvent and the suspension, propylene glycol, polyethylene glycol, vegetable oil such as olive oil, injectable ester such as ethyl oleate, and the like may be used.

The composition according to the present disclosure is administered in a pharmaceutically effective amount. In the present disclosure, the “pharmaceutically effective amount” refers to an amount enough to treat a disease at a reasonable benefit/risk ratio applicable to medical treatment. The effective dose level may be determined according to factors including the type and severity of a disease of a patient, the activity of a drug, the sensitivity to a drug, a time of administration, a route of administration, an excretion rate, duration of treatment, and simultaneously used drugs, and other factors well-known in the medical field.

The composition according to the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

The composition of the present disclosure may be administered to a subject through various routes. All methods of administration may be expected, and for example, the composition may be administered by oral administration, subcutaneous injection, intraperitoneal administration, intravenous injection, intramuscular injection, intrathecal injection, sublingual administration, buccal administration, rectal insertion, vaginal insertion, ocular administration, otic administration, nasal administration, inhalation, spraying through the mouth or nose, dermal administration, transdermal administration, etc.

The dose of the composition of the present disclosure is determined according to a type of drug as an active ingredient together with many related factors, such as a disease to be treated, an administration route, the age, sex, and weight of a patient, the severity of a disease, etc. Specifically, the effective amount of the composition according to the present disclosure may vary depending on the patient's age, sex, and weight, and may be generally administered in 0.001 to 150 mg per 1 kg of body weight, preferably 0.01 to 100 mg daily or every other day, or administered separately 1 to 3 times a day. However, since the dose may increase or decrease depending on a route of administration, the severity of a disease, sex, body weight, age, etc., the dose does not limit the scope of the present disclosure even in any way.

In the present disclosure, the “subject” refers to a subject in need of treatment for diseases, and more particularly, refers to mammals such as humans or non-human primates, mice, rats, dogs, cats, horses, pigs, sheep, and cow.

In the present disclosure, “administration” means providing a predetermined composition of the present disclosure to a subject by any suitable method.

In the present disclosure, “prevention” means any action of suppressing or delaying the onset of a target disease, “treatment” means any action of improving or beneficially changing a target disease and its resulting metabolic abnormality symptoms by administering the pharmaceutical composition according to the present disclosure, and “improvement” means any action of reducing parameters related to a target disease, for example, the degree of symptoms, by administering the composition according to the present disclosure.

### MODES OF THE INVENTION

Hereinafter, preferred Examples will be proposed in order to help in understanding of the present disclosure. However, the following Examples are just provided to more easily understand the present disclosure, and the contents of the present disclosure are not limited by the following Examples.

### EXAMPLES

### Example 1. Preparation of Cell-Derived Vesicles Containing Anchor Proteins (Anchor-CDVs)

A BioDrone platform using cell-derived vesicles (CDVs) intended to construct an effective drug delivery system by introducing targeting ligands or active cargos into cell-derived vesicles through genetic engineering. The CDVs to be used in the BioDrone platform have higher biocompatibility and lower immunogenicity than other nano-sized carriers due to physical and chemical similarities to extracellular vesicles (EVs), and also have a greater advantage than EVs in terms of productivity. For efficient modification of CDVs through genetic engineering, identification of anchor proteins that are stably and abundantly present in a CDV-specific manner is a very important basic step in the development of the BioDrone platform of the present disclosure.

### 1-1. Expression of Candidate Anchor Proteins Using Recombinant Vector

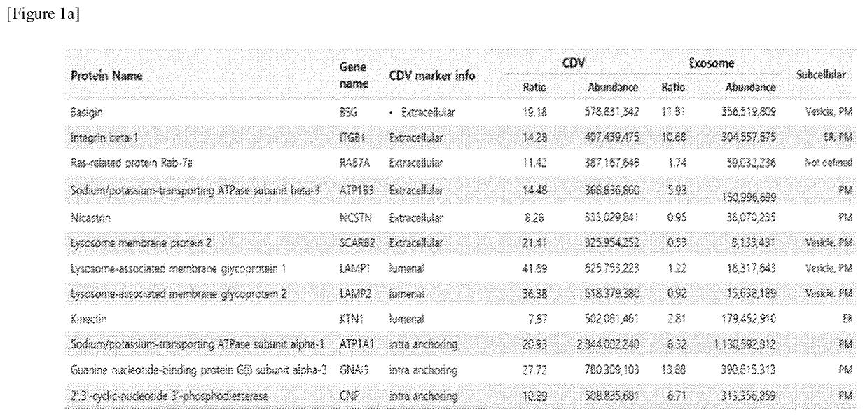

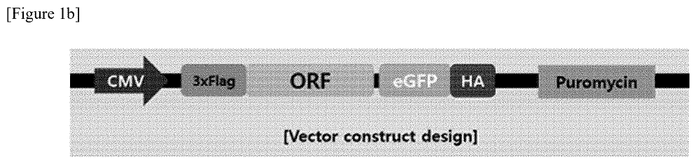

Through proteome analysis of cell-derived vesicles conducted in a previous study, 12 types of membrane proteins which were abundantly present in CDVs compared to cells and exosomes were selected as candidate anchor proteins (FIG. 1A). Fusion proteins were designed based on a total of 12 candidate membrane proteins and 2 comparative proteins (scaffold proteins selected from Codiak Biosciences). To rapidly produce cells overexpressing each of the proteins, a lentiviral vector was used. Cloning and preparation of viral particles were performed at VectorBuilder, and fusion protein genes with a design shown in FIG. 1B were cloned into a third-generation lentiviral vector system provided by VectorBuilder. To confirm the expression of each of the proteins and perform various other analyses, a 3×Flag tag was inserted at the N-terminus, and EGFP and HA tags were inserted at the C-terminus, respectively (FIG. 1B). A gene of interest (GOI) was designed to be located under a CMV promoter. In addition, EGFP was included for expression identification through fluorescence and quantitative analysis using ELISA. That is, since each candidate anchor protein was fused to GFP, the candidate anchor proteins may be detected indirectly through GFP.

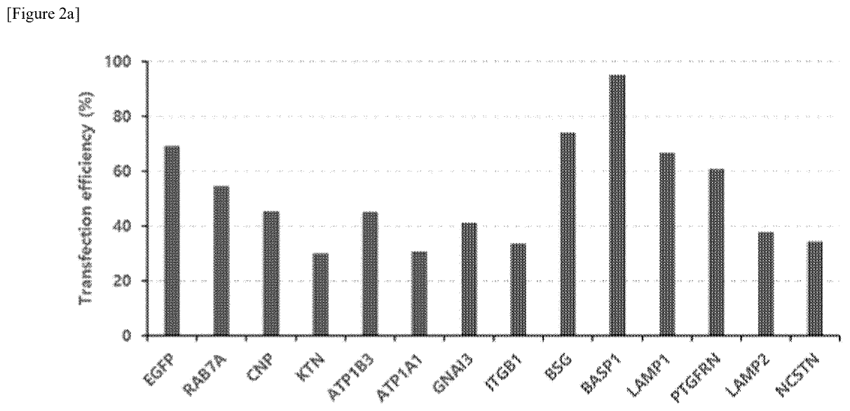

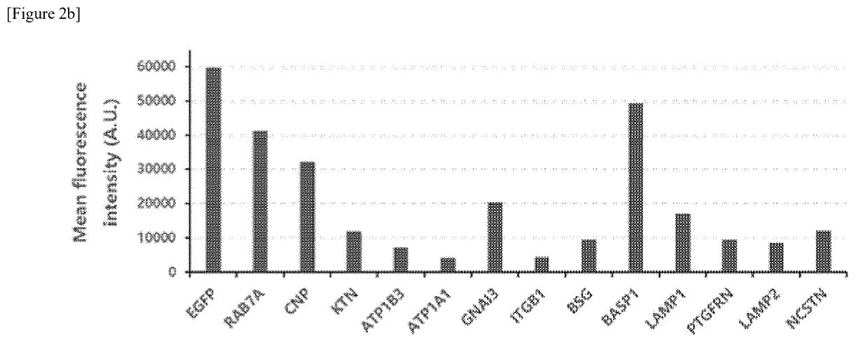

The expression of the fusion proteins using the constructed plasmid and virus particles was identified during the preparation process of VectorBuilder, but it was intended to verify the expression of the fusion protein itself. HEK293 cells, a cell line derived from human embryonic kidney, were transduced using the provided plasmid and Lipofectamine, and protein expression was verified by analyzing fluorescence expression. As a result, the expression of fluorescent proteins was confirmed in all plasmids, and the transduction efficiency was 30 to 95% depending on the plasmid (FIG. 2A). In addition, in the fluorescence intensity analysis, the fusion proteins of BASP1, RAB7A, CNP, and GNAI2 showed fluorescence intensities of approximately 20,000 or more to confirm high expression (FIG. 2B).

### 1-2. Generation of Stable Cell Lines Expressing Candidate Anchor Proteins

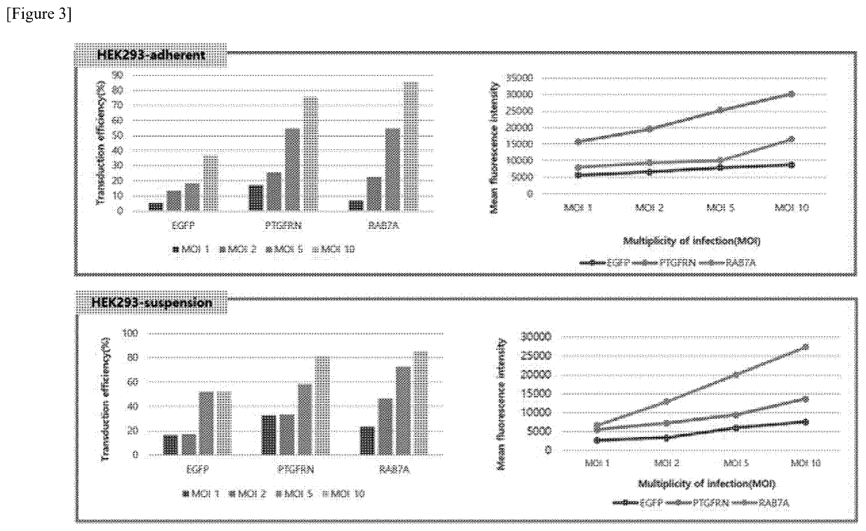

In Example, stable cell lines that stably expressed candidate anchor proteins were established. Before cell line generation through transformation, it was intended to determine the transduction efficiency according to a cell type (adherent or suspension) and a multiplicity of infection (MOI). The virus particles of EGFP, PTGFRN, and RAB7A fusion proteins, which had showed the transduction efficiency of 50% or more in Example 1-1, were treated to adherent and suspension HEK293 cells by varying MOI, and then the transformation efficiency was compared. As a result, no significant difference was observed according to a cell type, but increased efficiency and expression according to an MOI were confirmed (FIG. 3). Although a high MOI would be advantageous for short-term efficiency, it was determined to generate cell lines using adherent HEK293 cells, which are advantageous for removing cells that were not transformed after selecting MOI 5 due to concerns about viral cytotoxicity and treating antibiotics.

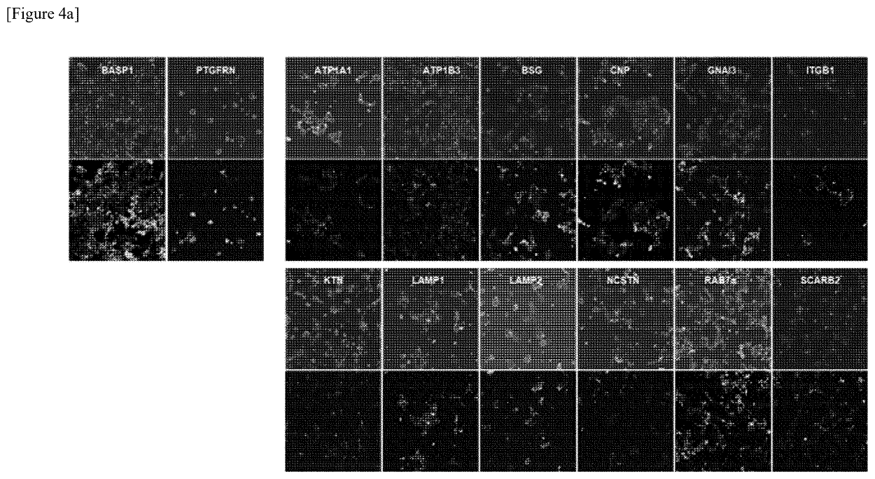

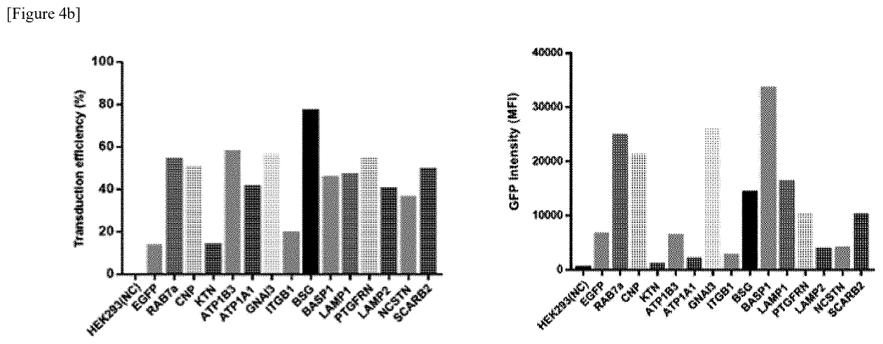

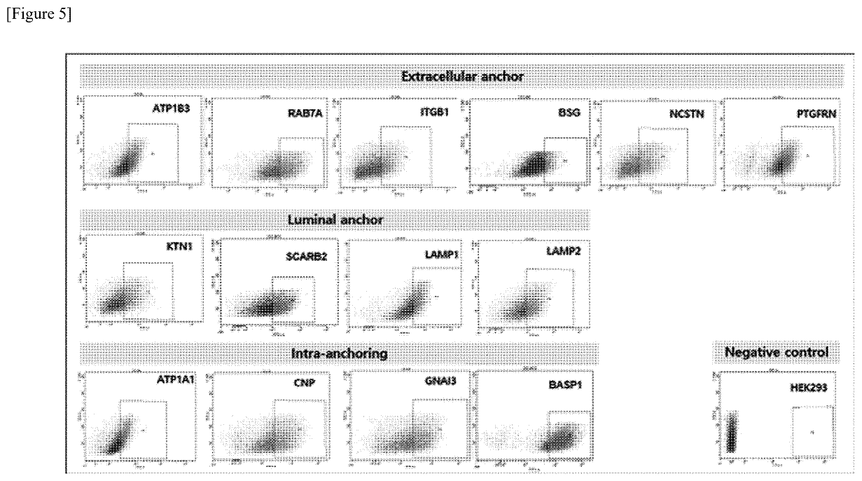

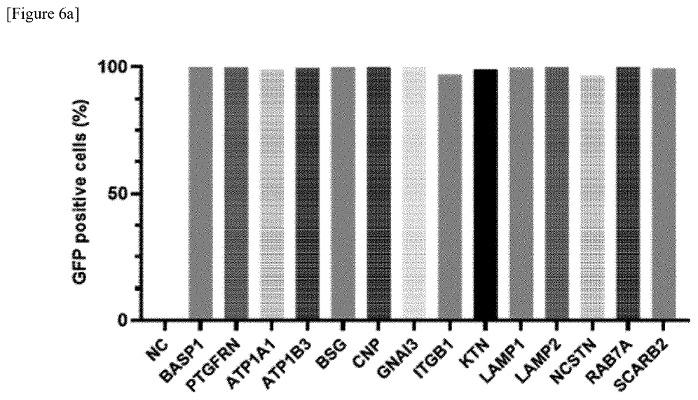

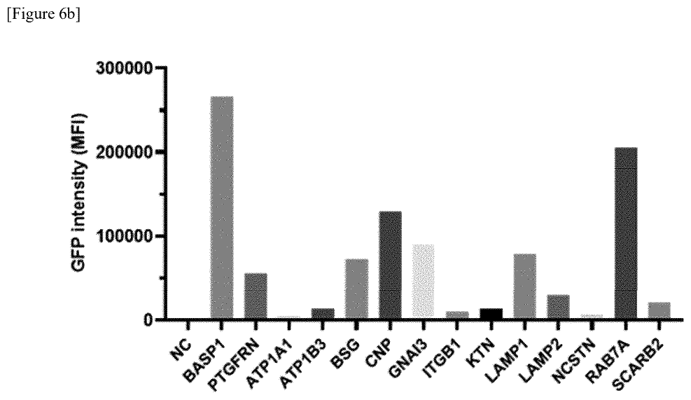

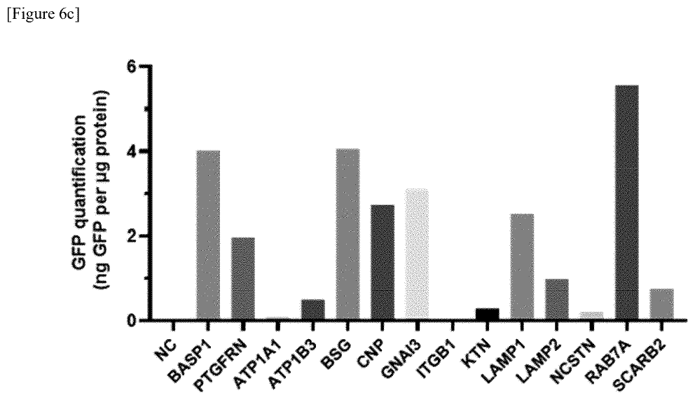

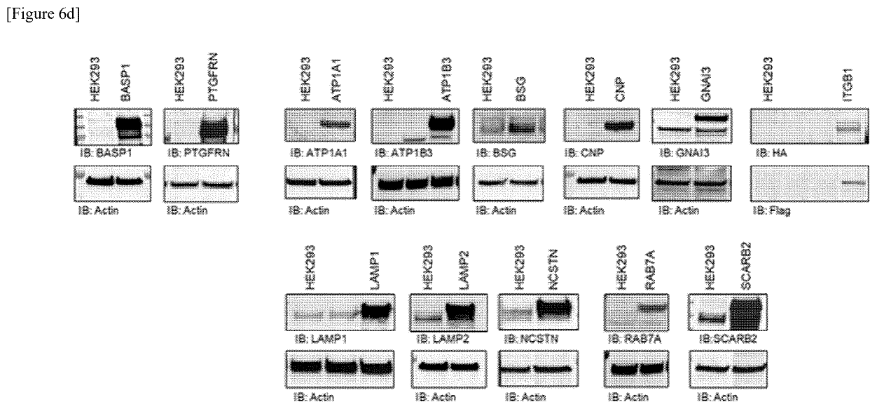

To generate the cell lines, virus particles of each fusion protein were treated, and the transduction efficiency was confirmed after 24 hours (FIGS. 4A and 4B). After 2 days, cell selection was started by treating 2 μg/mL of puromycin. Cells having resistance to antibiotics were incubated to 1×10⁷ cells, and then additional selection was performed using Cell Sorter. As shown in FIG. 5, cells with high fluorescence intensities were selectively obtained, and then incubated by adding antibiotics after 3 to 5 days for cell stabilization. As a result of analyzing the cells selected above, transformation efficiency of 90% or higher was confirmed in all cells, and thus it was confirmed that the selection of cells expressing the fusion proteins was successful (FIG. 6A). The expression levels of the fusion proteins varied for each cell, but cells expressing the fusion proteins BASP1, RAB7A, and CNP showed high fluorescence intensities, while ATP1A1 and NCSTN cells showed low fluorescence intensities (FIG. 6B).

The expression of the fusion protein was analyzed using GFP ELISA (Abcam, Cambridge, UK), and a different tendency was observed from the result of fluorescence intensity evaluation. For the fusion proteins of RAB7A, BSG, BASP1, GNAI3, CNP, LAMP1, and PTGFRN, high expression was observed, while for ITGB1, NCSTN, ATP1B3, KTN1, LAMP2, SCARB2, and ATP1A1, relatively low expression was observed (FIG. 6C). In addition, as results of performing Western blot analysis using antibodies that recognized each membrane protein for qualitative analysis, all membrane proteins were found to be overexpressed to different degrees, and thus it was confirmed that the fusion proteins (candidate anchor proteins) were expressed in a manner appropriate for the purpose of the study (FIG. 6D).

1-3. Characterization of CDVs Isolated from Anchor Protein Overexpressing Cells and Selection of Final Anchor Proteins

In Example, cell-derived vesicles (CDVs) were isolated from the transduced cells obtained through Example above, and their characteristics were confirmed. The transformed cells obtained through Example above were incubated to induce production of CDVs (anchor-CDVs), and the efficiency with which the candidate anchor protein expressed in each cell was delivered to CDV was confirmed. The ratio of GFP(+) vesicles was analyzed using a nanoparticle flow cytometer (NanoFCM, Inc.; Xiamen, China) and the amounts of fusion proteins were quantified using GFP ELISA.

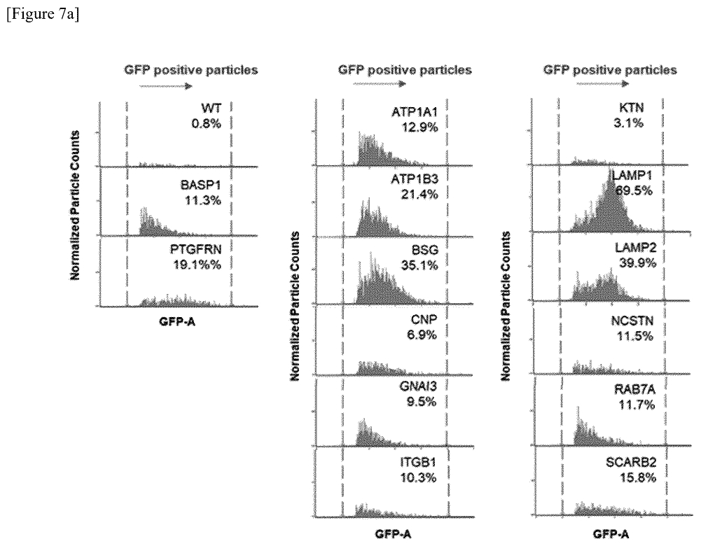

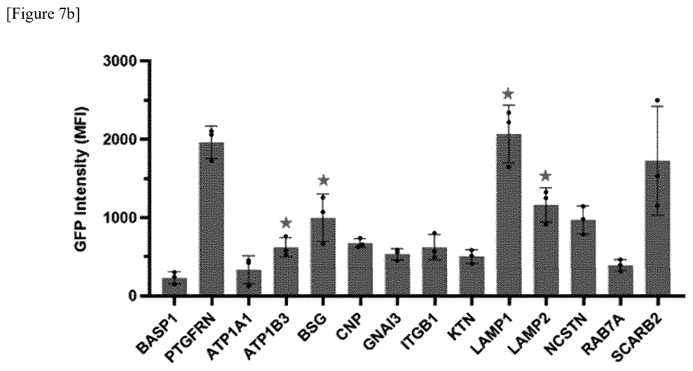

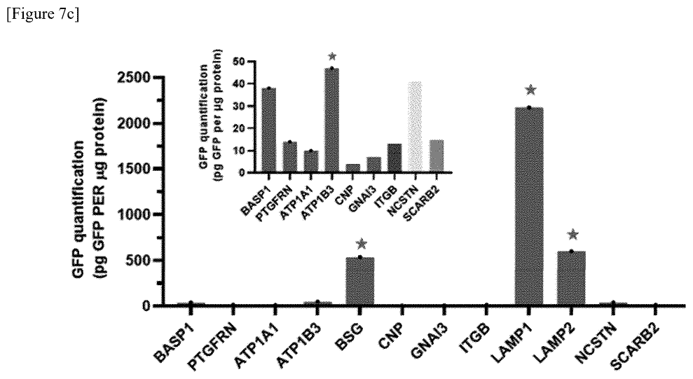

As a result, the ratio of GFP (+) vesicles varied from 3.1 to 69.5% depending on a fusion protein (FIG. 7A), and the amounts of fusion proteins were found to have an even greater variation. LAMP1, LAMP2, BSG, and ATP1B3 fusion proteins were introduced into CDVs in large quantities, but CNP and GNAI3, which were highly expressed in cells, were introduced into vesicles at low rates (FIGS. 7B and 7C).

PTGFRN and BASP1 from Codiak Biosciences, which were selected for comparison, were found to have lower amounts and introduction rates in CDVs than other membrane proteins, which was estimated that the introduction patterns were different because the generation principles of CDVs and extracellular vesicles (EVs) were different.

Based on these analysis results, it could be confirmed that membrane proteins specific to CDVs were required for engineering CDVs through transformation of parent cells, and ATP1B3, BSG, LAMP1, and LAMP2 were selected as anchor proteins for the BioDrone platform.

### 1-4. Identification of Final Anchor Proteins for BioDrone Platform

The final four types of BioDrone anchors selected through characterization of candidate anchor proteins of HEK-CDV are ATP1B3 and BSG, which are plasma membrane-derived proteins, and LAMP1 and LAMP2, which are known to be lysosome membrane-derived proteins.

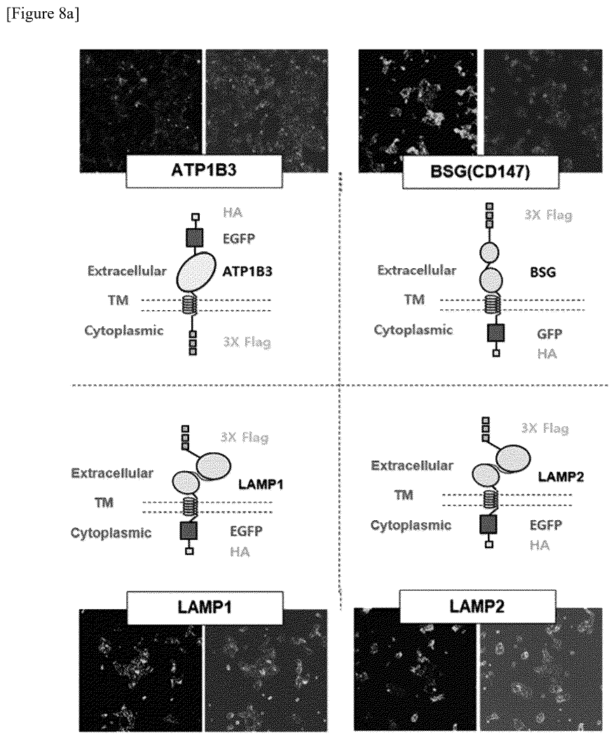

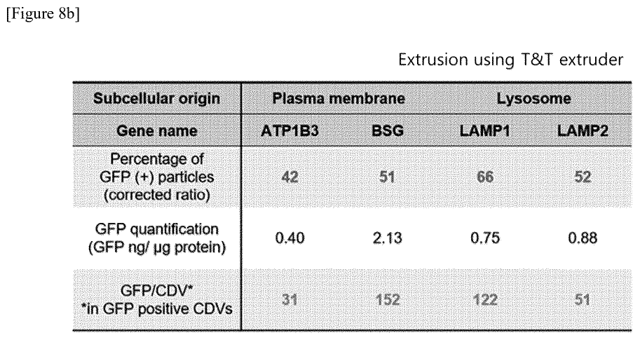

Stable expression of each selected anchor protein in HEK293 cells was confirmed over several generations, and a schematic diagram of each anchor-CDV based on the original topology was shown in FIG. 8A. Furthermore, based on the GFP (+) particle ratio from the nanoparticle flow cytometer analysis result and the GFP quantification value from the ELISA analysis result, the number of GFP molecules present per GFP (+) CDV particle (i.e., indirectly the number of anchor protein molecules) was theoretically calculated and represented (FIG. 8B). For example, it was confirmed that 152 and 122 GFP molecules were present per BSG-CDV and LAMP1-CDV, respectively.

### 1-5. Comparison of Anchor-CDV Characteristics According to CDV Extrusion Method

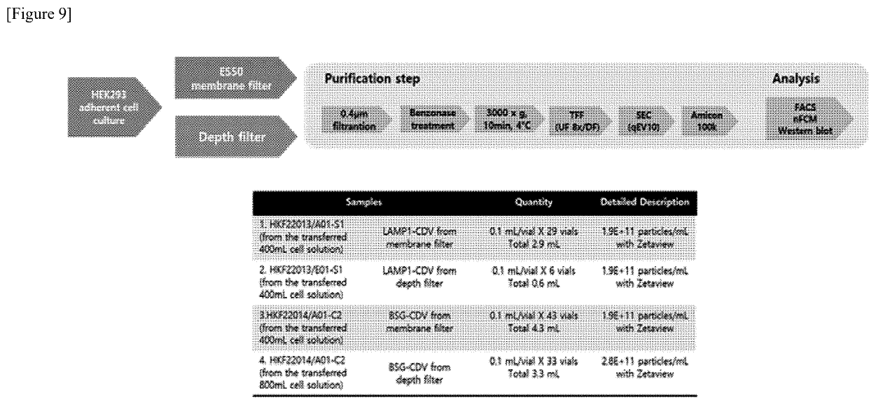

Although there were various cell-derived vesicle extrusion methods to be used in the BioDrone platform, tests for anchor protein selection were conducted on a small scale using a membrane filter (NanoSizer MINI Liposome Extruder). Although there was a risk to quickly test several candidate anchor proteins, efficient mass production of CDVs was required for the development and application of the BioDrone platform, and thus it was confirmed whether there were any changes in the characteristics of CDVs according to an extrusion method to derive the most appropriate CDV production method. The CDVs were extruded from two cell lines (HEK-BSG and HEK-LAMP1) overexpressing the selected anchor proteins using a membrane filter (ES-50) or a depth filter, respectively (FIG. 9), and it was confirmed whether the characteristics of cell-derived vesicles changed depending on an extrusion method.

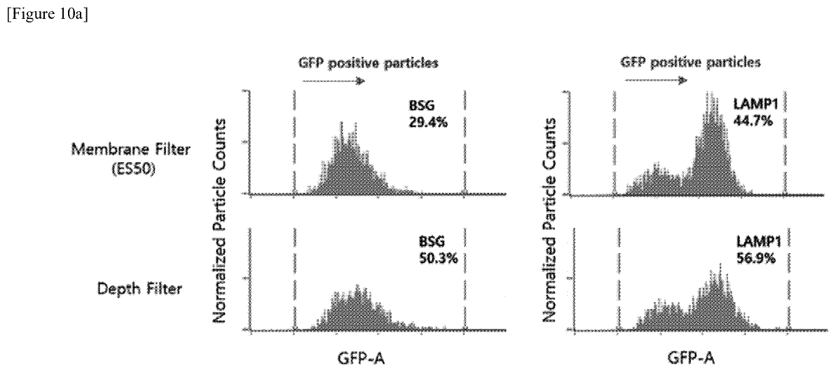

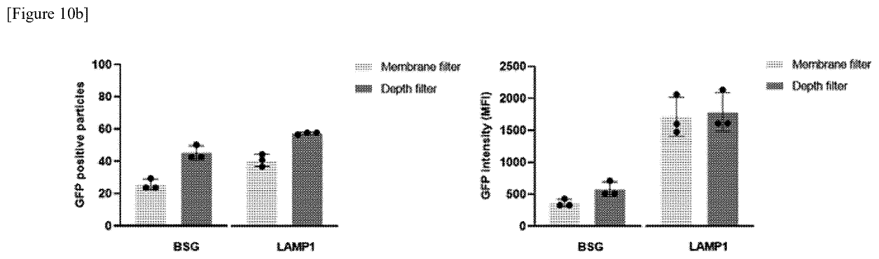

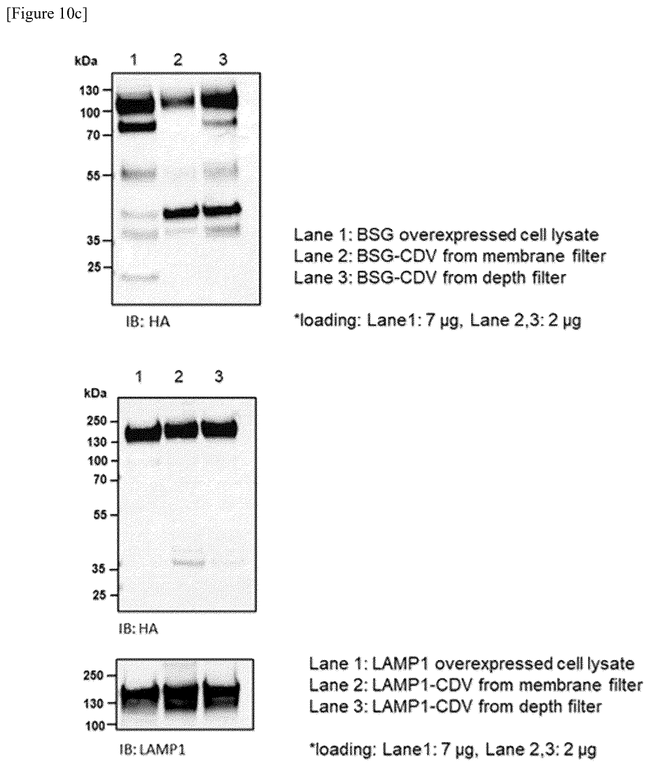

The distribution of GFP(+) particles at a single particle level was analyzed using CDVs extruded by different methods (FIGS. 10A and 10B), and the enrichment of anchor proteins was compared using Western blot (FIG. 10C). As a result, it was confirmed that the anchor proteins of the present disclosure showed a similar tendency regardless of the extrusion method and were present in each CDV. That is, although there may be a slight difference in the anchor protein introduction rate depending on a type of anchor protein, it was confirmed that there was no difference depending on the extrusion method.

1-6. Topology Analysis of Anchor Proteins Introduced into CDVs

In Example, topology analysis of anchor proteins was performed. To this end, proteinase K was treated on anchor-CDV to remove the extra-part of the protein present in the CDV membrane, and then the topology of each protein was confirmed by Western blot.

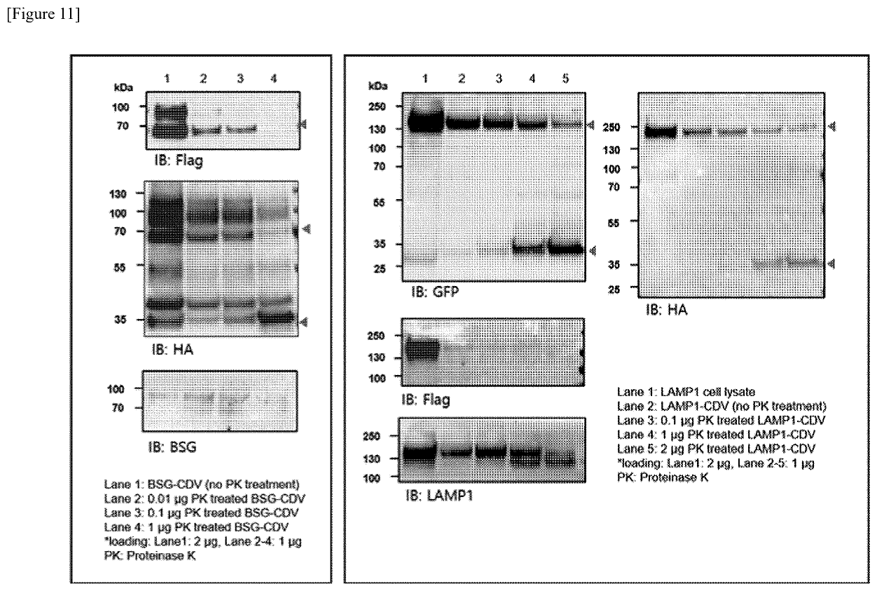

When designing the plasmid construct, different tags (Flag tag and HA tag, respectively) were introduced at the N- or C-terminal to be used for topology analysis of the anchor proteins. The overall experimental outline is to induce digestion of the extra-vesicular part of the membrane protein by treatment with proteinase K, and then to determine the degree to which the anchor proteins are detected with antibodies against different tags introduced at the N- or C-terminal. When the extra-vesicular part of the anchor protein is the N-terminal, the level of the anchor protein detected with anti-Flag decreases when treated with proteinase K, and when the extra-vesicular part is the C-terminal, the level of the anchor protein detected with anti-HA decreases. After treatment with proteinase K in BSG-CDV and LAMP1-CDV, when the proteins were detected with anti-HA, the detection level of proteins corresponding to the original size decreased, and after protein digestion outside the vesicles, the detection level of small-sized proteins remaining therein increased. That is, it was confirmed that BSG and LAMP1 maintained their original topology even after CDV introduction (FIG. 11). The results show that the characteristics of the anchor proteins of the present disclosure do not change even when introduced into CDVs.

In conclusion, through Examples, ATP1B3, BSG, LAMP1, and LAMP2 were selected as four types of anchor proteins for engineering CDVs, and it was confirmed that the anchor proteins were stably present on CDV particles. Therefore, by using these anchor proteins, targeting ligands may be fused or therapeutic cargos may be introduced to be introduced into CDVs, and thus, may be utilized in various ways for effective and efficient CDV engineering for drug delivery.

### Example 2. Development of HER2-Targeted Anchor-CDVs

In order to verify whether the anchor-CDVs according to the present disclosure may be utilized as a drug delivery system, targeting ligands or active cargos were expressed in the form of fusion proteins with anchor proteins and introduced into the CDVs, and then it was intended to verify whether the CDV targeted the target cell and whether the CDV was bound to or absorbed in the cell. Accordingly, in Example, CDVs targeting human epidermal growth factor receptor2 (HER2) were prepared and the effect thereof was confirmed. To impart the targeting property to CDV, a single-chain variable fragment (scFv) site of trastuzumab, which was a HER2 protein-specific antibody, was used as a targeting ligand. A cell line overexpressing scFv (scTTZ) of trastuzumab was constructed, and anchor-CDVs introduced with HER2-targeting scFv were obtained therefrom, and then the tumor-targeting ability of the anchor-CDVs was evaluated in a HER2-positive tumor animal model.

### 2-1. Preparation of Trastuzumab-Overexpressing Cell Lines Using Lentivirus-Based Transduction

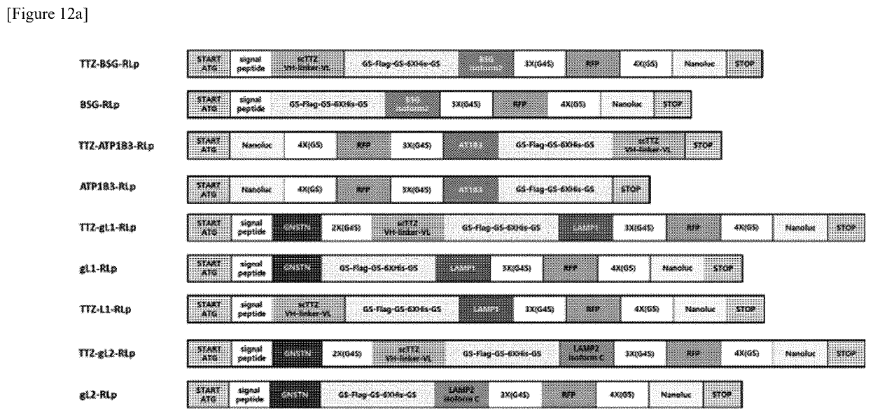

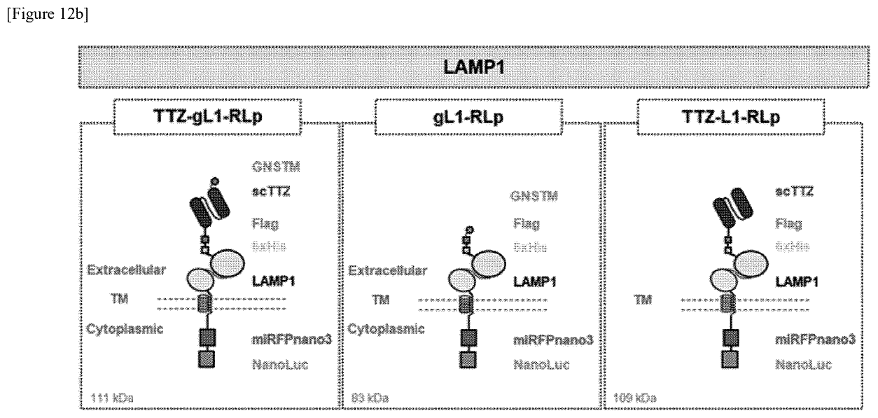

In the previous Examples, it was attempted to introduce targeting ligands into CDVs using four membrane proteins selected as BioDrone anchor proteins. To this end, first, a cell line expressing scFv (scTTZ) of trastuzumab as the HER2 antibody was prepared. For the four selected membrane proteins BSG, ATP1B3, LAMP1, and LAMP2, since the membrane proteins were abundantly present in CDVs compared to cells or exosomes, it was expected to be more stably introduced into CDVs than when overexpressing targeting ligands using these anchors. To prepare cell lines using lentiviral transduction, a vector construct capable of expressing the anchor and trastuzumab scFv (scTTZ) in a fused form was designed to prepare lentiviral particles (FIGS. 12A and 12B).

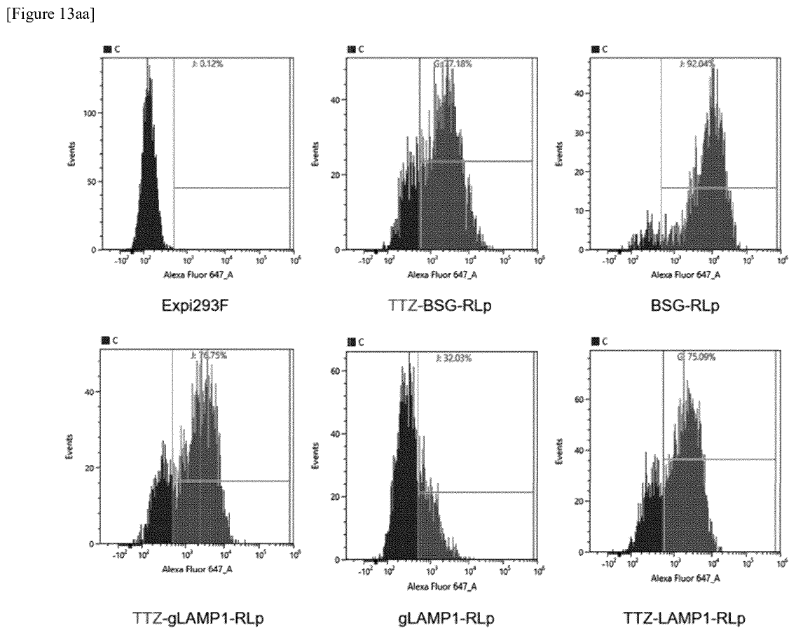

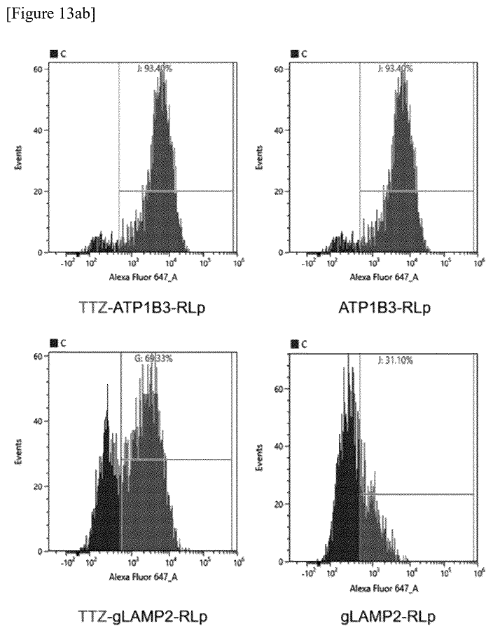

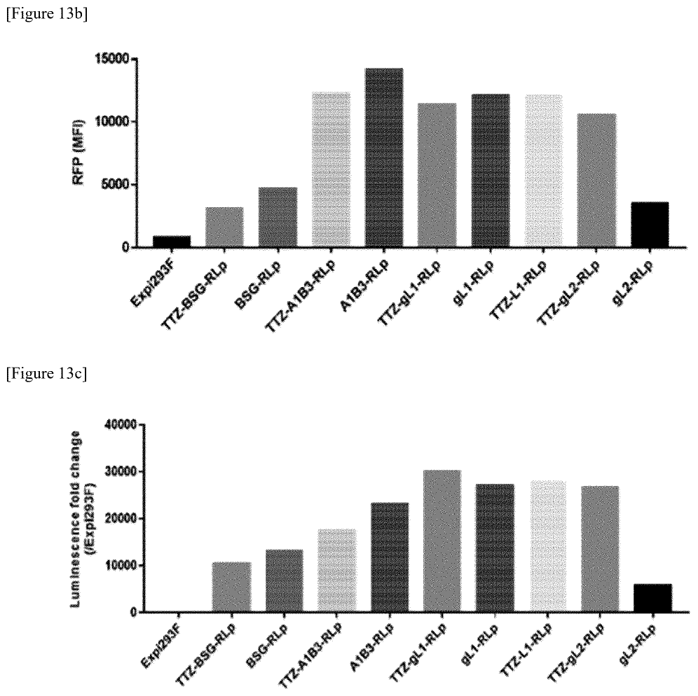

A total of 9 cell lines, including a control group, were prepared through transduction with the prepared lentiviral particles. After 24 hours of transduction, miRFPnano3 fluorescence was observed through a fluorescence microscope, and as a flow cytometry result, it was confirmed that approximately 30 to 95% of cells exhibited fluorescence (FIG. 13A). The transduction cells were incubated for about a week, and when the viability was approximately 90% and 30 mL was secured at a concentration of 5.0×10⁵ cells/mL, cells expressing scTTZ and anchor proteins could be selected by treating puromycin at 2 μg/mL. The cell condition was observed for about 3 weeks and the puromycin concentration was adjusted from 2 μg/mL to 5 μg/mL, and the expression of 90% of higher was confirmed in all cell lines.

In addition, as the result of comparing the miRFPnano3 fluorescence intensity and luciferase activity for the selected cells, it was shown that the fluorescence intensity and luciferase activity between the cells had a similar tendency (FIGS. 13B and 13C). That is, the expression levels of scTTZ and the anchor proteins were indirectly evaluated through fluorescence intensity analysis, and it was confirmed that both miRFPnano3 and luciferase fused to the C-terminus were stably expressed.

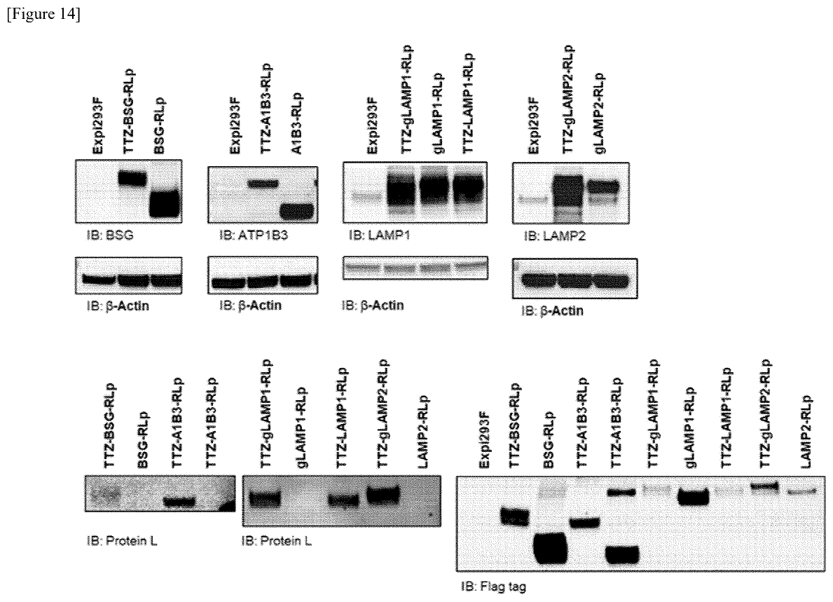

Next, the expression of anchor proteins, scTTZ, and Flag tag was analyzed through Western blot (FIG. 14). As the previous results, the expression levels varied for each cell, but when the protein size and band thickness were confirmed, it was confirmed that all proteins were well expressed. When the miRFPnano3 and luciferase activity results were considered together, it was confirmed that among the anchor proteins, particularly, LAMP1 and LAMP2 stably expressed the scTTZ fusion protein.

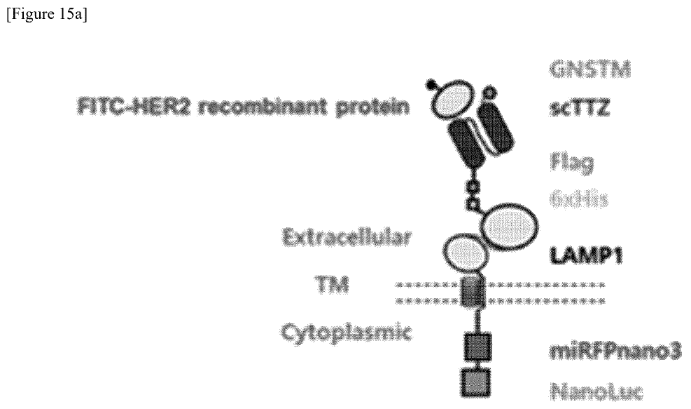

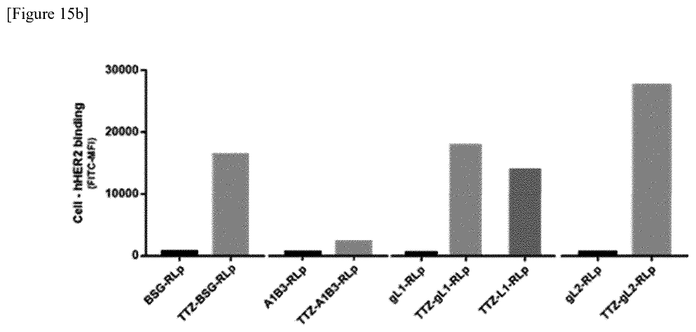

To confirm whether scTTZ expressed in each cell bound to the target protein, binding to FITC-labeled recombinant human HER2 was confirmed (FIG. 15A). When cells with only anchors without scTTZ were used as a control group, it was confirmed that in all four anchor proteins, the FTIC fluorescence signals were increased by HER2 binding to scTTZ (FIG. 15B). In particular, although LAMP1 and LAMP2 were lysosomal membrane proteins, the LAMP1 and LAMP2 were expressed in large amounts on the plasma membrane when overexpressed, and it was confirmed that HER2 was bound in large quantities even without cell permeabilization.

2-2. Preparation of Anchor-CDVs Introduced with Trastuzumab (scFv)

CDVs were produced by extrusion using ES-50 for cell lines expressing scTTZ and anchor proteins. The CDVs could be obtained at a final concentration of 5.0×10¹⁰ to 1.5×10¹¹ ps/mL through SEC purification and Amicon concentration. The particle concentration and volume (yield) at each CDV production step were shown in Table 1.

TABLE 1

| Step | Cell and CDV concentration and volume | |
|---|---|---|
| Cell | Cell suspension concentration | 1.0 × 10⁶ |
| | (cells/mL) | |
| | Cell suspension volum (mL) | 100 |
| Crude-CDV | Extruded CDV concentration | 1.0 to 2.8 × 10¹⁰ |
| | (ps/mL) | |
| | Extruded CDV volume (mL) | 85 to 90 |
| Amicon | Concentrated CDV concentration | 0.5 to 1.0 × 10¹² |
| Concentration | (ps/mL) | |
| | Concentrated CDV volume (mL) | 2 |
| SEC | Purified CDV concentration | 0.5 to 1.0 × 10¹¹ |
| Purification | (ps/mL) | |
| | Purified CDV volume (mL) | 8 |

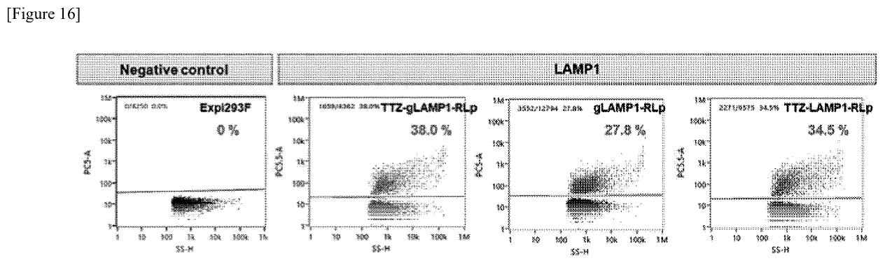

The extruded CDVs were analyzed using a nanoparticle flow cytometer to confirm the ratio of miRFPnano3 positive particles among CDVs. The fluorescence ratio varied slightly depending on an anchor was 4 to 38%, which was slightly different, but it was confirmed that all of the anchor-CDVs expressed the fluorescence. In particular, it was confirmed that the introduction rate of ssTTZ was high when LAMP1 was used as the anchor protein, and the introduction rate of ssTTZ into CDV was further increased when glycosylated LAMP1 (gLAMP1) was used (FIG. 16). Therefore, subsequent experiments were conducted using LAMP1-CDV and gLAMP1-CDV as representative examples.

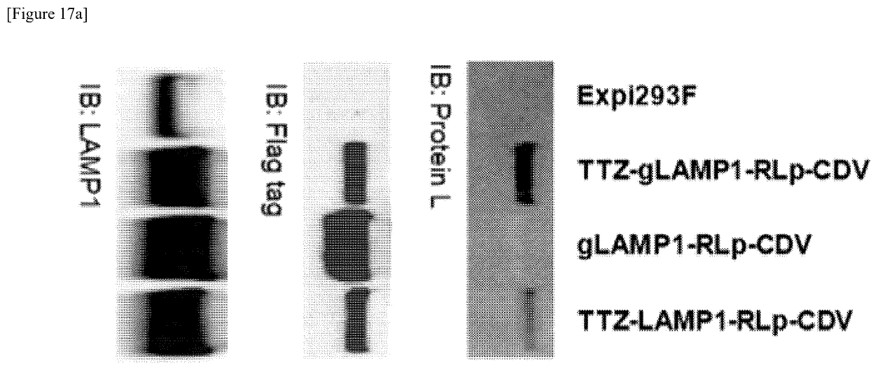

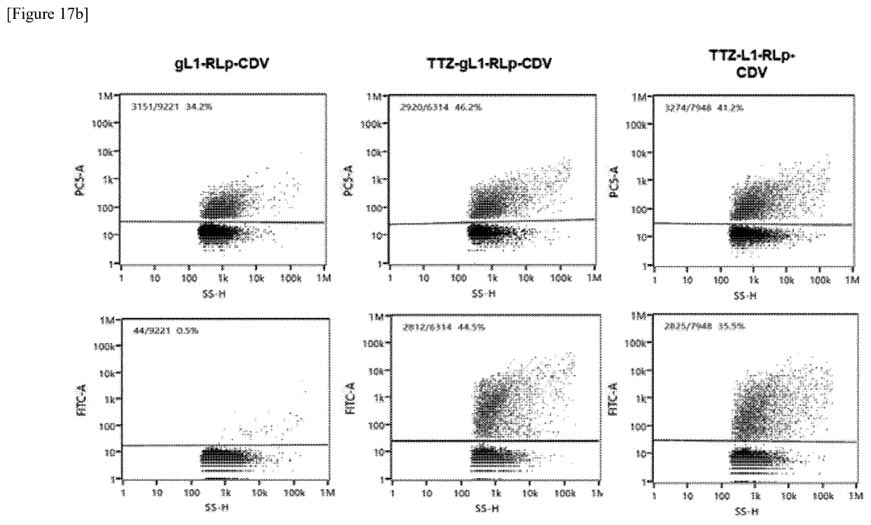

The expression of the CDV fusion protein with scTTZ anchored by LAMP1 was confirmed by Western blot (FIG. 17A), and binding to recombinant HER2 (FITC-HER2) was confirmed by a nanoparticle flow cytometer (FIG. 17B). A thick band of LAMP1 was observed even in CDV, and when confirmed with protein L, a relatively thicker band was confirmed in CDV using glycosylated LAMP1 as an anchor. Even in the Nanoparticle flow cytometer analysis result, it was also shown that CDV using glycosylated LAMP1 expressed miRFPnano3 (using PC₅ fluorescence) at a 5% higher rate than AMP1. When FITC-labeled HER2 was bound to CDV, only 0.5% of CDV in a control gLAMP1-CDV showed fluorescence, which was considered to be non-specific binding. In contrast, FITC fluorescence was identified in 44.5% and 35.5% of CDVs in scTTZ-gLAMP1 and LAMP1-CDV, respectively, which indicated that most CDVs with scTTZ effectively bound to HER2, when considering the ratio of miRFPnano3 positive CDVs.

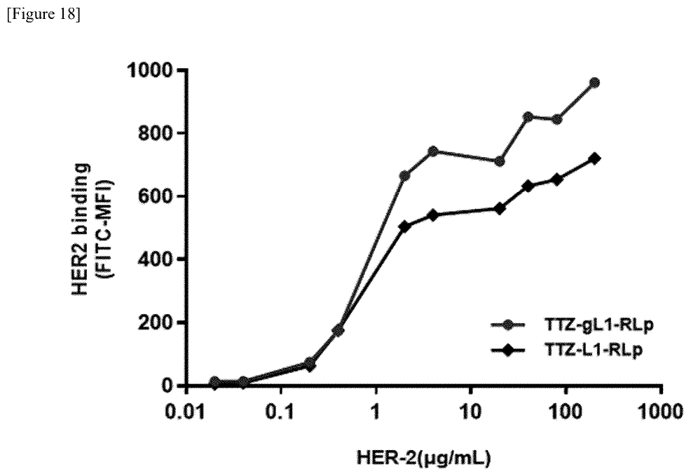

To confirm a difference of scTTZ-CDV depending on the presence or absence of glycosylation of LAMP1, CDVs were attached to aldehyde/sulfate latex beads, and reacted with FITC-HER2 protein at various concentrations to compare EC50 values (FIG. 18). First, both types of CDVs were able to confirm concentration-dependent HER2 binding, and the EC50 was calculated using this, and as a result, 0.94 μg/mL for gLAMP1-CDV and 0.88 μg/mL for LAMP1-CDV were confirmed. Considering the ratio of scTTZ according to each CDV, it was considered that a difference in EC50 was not significant, and no effect depending on glycosylation was observed.

2-3. In Vitro Evaluation of Anchor-CDVs Introduced with Trastuzumab (scFv)

### (1) Target Cell Binding Analysis

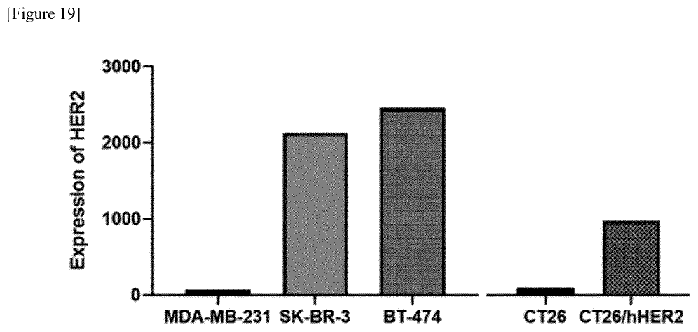

Before evaluating the binding between scTTZ-CDV and HER2-expressing cells, the expression levels of HER2 were confirmed in three types of breast cancer cells, BT-474, SK-BR-3, and MDA-MB-231, and CT26 and CT26/hHER2 cells. BT-474 and SK-BR-3 cells are known as high HER2 expression cell lines, but the doubling time is 2 to 3 days, making it difficult to create a tumor animal model due to a slow proliferation rate. As an alternative, CT26/hHER2, which was prepared to express human HER2 in mouse-derived cells, was used. As shown in FIG. 19, it was confirmed that HER2 was not expressed in HER2-negative cell lines, MDA-MB-231 and CT26 cells. On the other hand, in the HER2-positive cell lines BT-474 and SK-BR-3, at least 99% of cells expressed HER2, and in CT26/hHER2, at least 90% of cells expressed hHER2. In addition, it was confirmed that hHER2 was expressed at a level approximately 2 times higher in BT-474 and SK-BR-3 than CT26/hHER2.

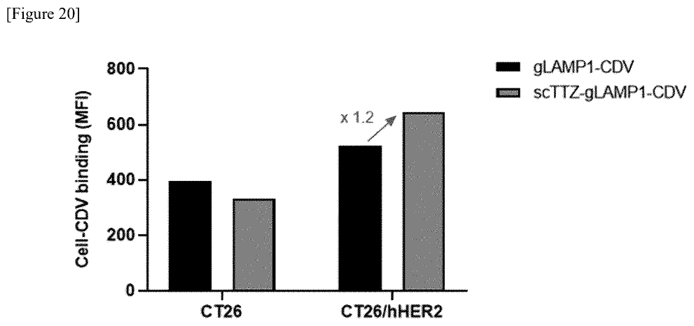

Next, the binding of CDV to HER2 by introducing scTTZ for CT26/hHER2 was confirmed. CFSE-fluorescently labeled scTTZ-gLAMP1-CDV and a control gLAMP1-CDV were co-cultured with CT26/hHER2 or control CT26 cells, and the relative binding degree was compared by detecting the fluorescence intensity using flow cytometry. As a result, as shown in FIG. 20, no difference in fluorescence was observed between the two types of CDVs regardless of the presence or absence of scTTZ in CT26 cells, as a HER2-negative cell line. On the other hand, in the case of CT26/hHER2 cells, the histogram peak of flow cytometry shifted by approximately 6% due to the binding of scTTZ-gLAMP1-CDV, and thus it was confirmed that scTTZ-gLAMP1-CDV bound to the cells.

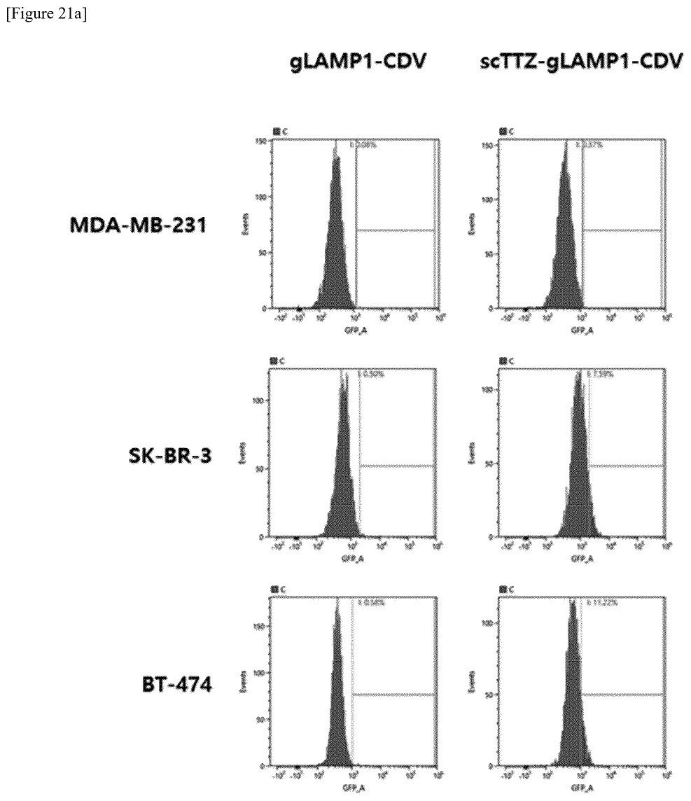

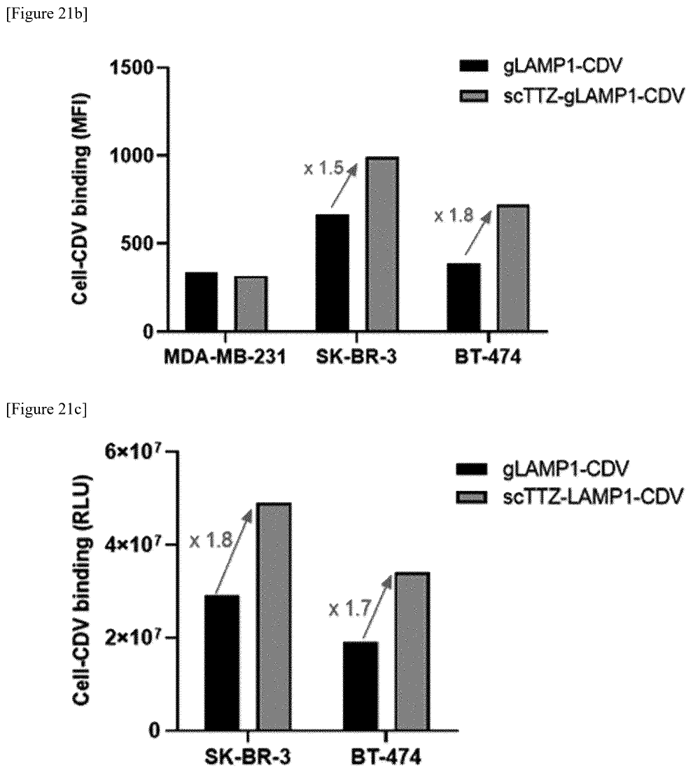

Next, the binding of scTTZ-gLAMP1-CDV was evaluated using HER2 high-expressing cells. HER2 high-expressing cell lines BT-474 and SK-BR-3 were used, and HER2 negative cell line, MDA-MB-231 cells were used as a control. As a result, no difference in binding between CDVs was observed for MDA-MB-231 cells, whereas a peak shift of approximately 7.5 to 11% was observed for scTTZ-gLAMP1-CDV compared to the control CDV in BT-474 and SK-BR-3 (FIG. 21A). As a result of quantifying the fluorescence intensity of CDVs bound to the cells, it was confirmed that approximately 1.5-fold and 1.8-fold more CDVs were bound to SK-BR-3 and BT-474 cells, respectively (FIG. 21B). In addition, it was confirmed that the binding of the HER2 high-expressing cell line to scTTZ-LAMP1-CDV was 1.8-fold and 1.7-fold enhanced for SK-BR-3 and BT-474 cells, respectively, when comparing the activity using luciferase fused to CDV rather than the fluorescence intensity (FIG. 21C). It is expected that the binding affinity for such a target protein may be further improved by using full-length antibodies rather than scFv. The results show that the anchor-CDVs of the present disclosure may stably introduce antibody-based drugs into CDVs through the anchor proteins, and effectively target the target cells based on the antibodies.

(2) Analysis of Uptake into Target Cells

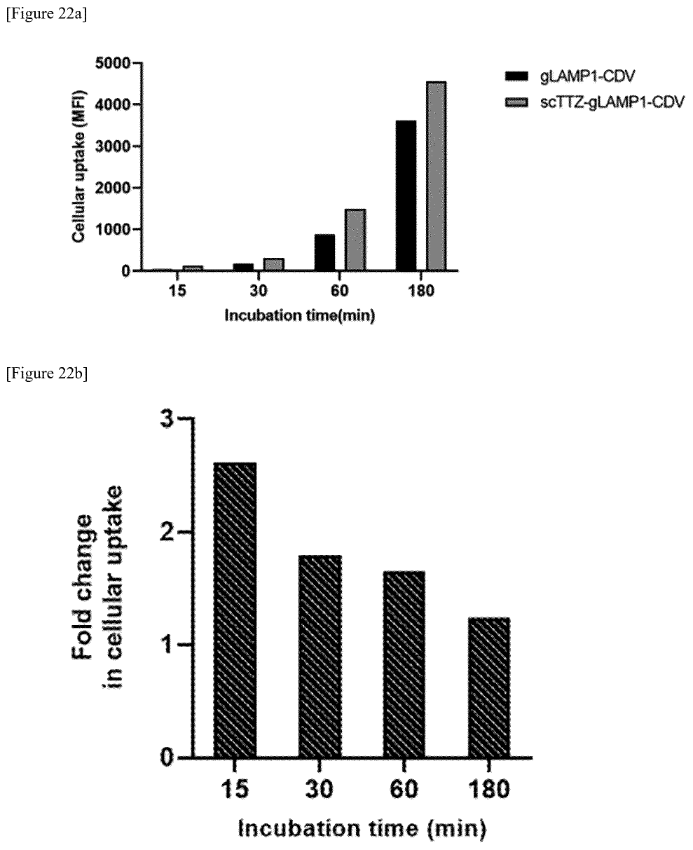

Next, in order to confirm whether ssTTZ increases the uptake of CDVs into cells, BT-474 cells were treated with gLAMP1-CDV and scTTZ-LAMP1-CDV stained with DiO, and then the uptake patterns into cells were confirmed. To determine a difference in the amount absorbed into cells at 15, 30, 60, and 180 minutes after incubating the cells, DiO fluorescence was compared by flow cytometry, and the results were analyzed by considering the relative fluorescence intensity for each sample (FIG. 22A). As a result, it was confirmed that scTTZ-LAMP1-CDV was absorbed into cells in amounts approximately 2.62 times greater at 15 minutes, 1.79 times greater at 30 minutes, 1.65 times greater at 1 hour, and 1.24 times greater at 3 hours than the control group. In addition, the difference was greatest in first 15 minutes and then gradually decreased over a culture time (FIG. 22B), which suggested that initially, uptake into cells increased due to scTTZ introduced into CDV and binding to HER2 on the cell surface (receptor-mediated endocytosis), and then over time, CDV was absorbed into cells regardless of the presence or absence of scTTZ.

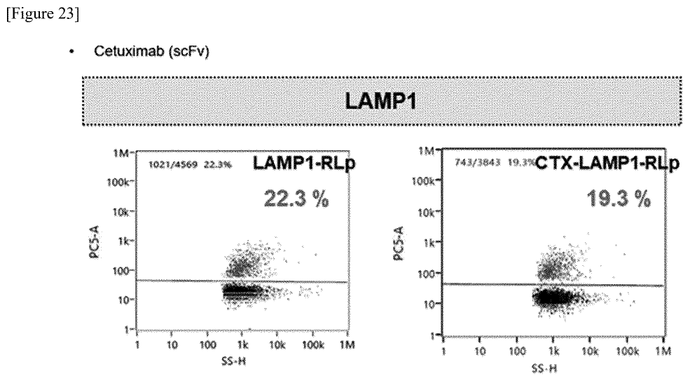

In summary, it was confirmed that a targeting ligand capable of targeting a specific protein may be effectively introduced into CDV via the anchor protein of the present disclosure, and that the CDV introduced with the targeting ligand may bind to or be absorbed in by cancer cells expressing the target protein, which suggested that the anchor-CDV of the present disclosure may be utilized as an effective drug delivery system. In particular, the introduction of targeting ligands into CDVs using the anchor proteins was confirmed not only by scFv of trastuzumab but also by scFv of cetuximab (FIG. 23), and thus it is expected that the anchor-CDVs of the present disclosure will be useful for loading and delivering various targeting ligands.

### Example 3. Development of Anchor-CDV Introduced with GFP

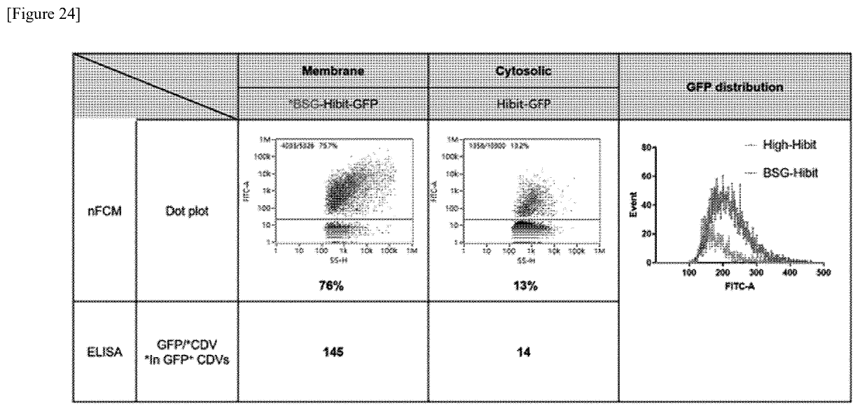

In order to verify the usability of the anchor-CDVs according to the present disclosure, an anchor-CDV introduced with a fluorescent protein GFP was prepared and then it was confirmed whether the fluorescent protein was normally introduced into the CDV. Accordingly, a vector construct capable of expressing GFP in a fused form with BSG, one of the anchor proteins of the present disclosure, was fabricated, lentiviral particles were prepared, and then HEK293 cells were transduced with the lentiviral particles. As a control group, cells overexpressing only GFP without anchor proteins were used. After extruding CDVs from each cell, the fluorescence of CDVs was detected using a nanoparticle flow cytometer and ELISA. As a result, in the case of CDVs introduced with BSG-GFP, GFP fluorescence was detected in at least 75% of CDVs, whereas in the case of CDVs introduced with only GFP, GFP fluorescence was detected in only about 13% of CDVs (FIG. 24). The results show that the anchor proteins of the present disclosure are used to more effectively introduce an active ingredient or cargo into CDV.

The aforementioned description of the present disclosure is to be exemplified, and it may be understood by those skilled in the art that the technical spirit or required features of the present disclosure may be easily modified in other detailed forms without changing. Therefore, it should be appreciated that the aforementioned exemplary embodiments are illustrative in all aspects and are not restricted.

TABLE 2

| CLASSSIFICATION | SEQUENCE | SEQ ID NO: |
|---|---|---|
| ATP1B3 | TKNEKKSLHQSLAEWELFTYNPTTGEPLGRTAKSWGLILLF | 1 |
| | YLVFYGFLAALPSPTMWVMLQTLNDEVPKYRDQIPSPGL | |
| | MVFPKPVTALEYTESRSDPTSYAGYIEDLKKFLKPYTLEEQ | |
| | KNLTVCPDGALFEQKGPVYVACQFPISLLQACBGMNDPDF | |
| | GYSQGNPCILVKMNRIIGLKPEGVPRIDCVSKNEDIPNVAVY | |
| | PHNGMIDLKYFPYYGKKLHVGYLQPLVAVQVSFAPNNTG | |
| | KEVTVECKIDGSANLKSQDDRDKFLGRVMFKITARA | |
| BSG | AAGTVFTTVEDLGSKILLTCSINDSATEVTGHRWLKGGVV | 2 |
| | LKEDALPGQKTEFKVDSDDQWGEYSCVFLPEPMGTANIQL | |
| | HGPPRVKAVKSSEHINEGETAMLVCKSESVPPVTDWAWY | |
| | KITDSEDKALMNGSESRPFVSSSQGRSELHIENLNMEADP | |
| | GQYRCNGTSSKGSDQAIITLRVRSHLAALWPFLGIVAEVLV | |
| | LVTIFTYEKRRKPEDVLDDDDAGSAPLKSSGQHQNDKGKN | |
| | VRQRNSS | |
| LAMP1 | AMFMVKNGNGTACIMANFSAAFSVNYDTKSGPKNMTFDL | 3 |
| | PSDATVVLNRSSCGKENTSDPSLVIAFGRGHTLTLNFTRN | |
| | ATRYSVQLMSFVYNLSDTHLFPNASSKEIKTVESITDIRAD | |
| | IDKKYRCVSGTQVHMNNVTVTLHDATIQAYLSNSSPSRGE | |
| | TRCEQDRPSPTTAPPAPPSPSPSPVPKSPSYDKYNVSGTN | |
| | GTCLLASMGLQLNLTYERKDNTTVTRLLHINPNKTSASGS | |
| | CGAHLVTLELHSEGTTVLLFQFGMNASSSRFFLQGIQLNTI | |
| | LPDARDPAFKAANGSLRALQATVGNSYKCNAEEHVRVTK | |
| | APSVNIPKVWVQAFKVEGGQPGSVEECLLDENSMLIPIAVG | |
| | GALAGLVLIVLIAYLVGRERSHAGYQTI | 4 |
| LAMP2 | LELNLTDSENATCLYAKWQMNFTVRYETTNKTYKTVTIS | |
| | DHGTVTYNGSICGDDQNGPKIAVQFGPGFSWIANFTKAAS | |
| | TYSIDSVSFSYNTGDNTTFPDAEDKGILTVDELLAIRIPLND | |
| | LFRCNSLSTLEKNDVVQHYWDVLVQAFVQNGTVSTNEPL | |
| | CDKDKTSTVAPTIHTTVPSPTTTPTPKEKPEAGTYSVNNG | |
| | NDTCLLATMGLQLNITQDKVASVININPNTTHSTGSCRSH | |
| | TALLRLNSSTIKYLDFVFAVKNENRFYLKEVNISMYLVNG | |
| | SVPSIANNNLSYWDAPLGSSYMCNKEQTVSVSGAPQINTF | |
| | DLRVQPFNVTQGKYSTABECSADSDLNFLIPVAVGVALGF | |
| | LITVVFISYNIGERKSRTGYQSY | |
| trastuzumab scFv | EVQLVESGGGLVQPGGSLRLSCAASGENIKDTYIHWYRQA | 5 |
| | PGKGLEWVARTYPINGYTRYADSVKGRFTISADTSKNTAY | |
| | LQMNISLRAEDTAVYYCSRWGGDGFYAMDYWGQGTLVTV | |
| | SSGGSSRSSSSGGGGSGGGGDIQMTQSPSSLSASVGDRVTI | |
| | TCRASQDVNTAVAWYQQKPGKAPKLLIYSASFLYSGVPSR | |
| | FSGSRSGTDFTLTISSLQPEDPATYYCQQHYTTPPTFGQG | |
| | TKVEIK | |
| ATP1B3 | ACGAAGAACGAGAAGAAGTCCCTCAACCAGAGCCTGGC | 6 |
| | CGAGTGGAAGCTCTTCATCTACAACCCGACCACCGGAG | |
| | AATTCCTGGGGCGCACCGCCAAGAGCTGGGGTTTGATCT | |
| | TGCTCTTCTACCTAGTTTTTTATGGGTTCCTGGCTGCAC | |
| | TCTTCTCATTCACGATGTGGGTTATGCTTCAGACTCTCA | |
| | ACGATGAGGTTCCAAAATACCGTGACCAGATTCCTAGC | |
| | CCAGGACTCATGGTTTTTCCAAAACCAGTGACCGCATTG | |
| | GAATATACATTCAGTAGGTCTGATCCAACTTCGTATGCA | |
| | GGGTACATTGAAGACCTTAAGAAGTTTCTAAAACCATA | |
| | TACTTTAGAAGAACAGAAGAACCTCACAGTCTGTCCTG | |
| | ATGGAGCACTTTTTGAACAGAAGGGTCCAGTTTATGTTG | |
| | CATGTCAGTTTCCTATTTCATTACTTCAAGCATGCAGTG | |
| | GTATGAATGATCCTGATTTTGGCTATTCTCAAGGAAACC | |
| | CTTGTATTCTTGTGAAAATGAACAGAATAATTGGATTAA | |
| | AGCCTGAAGGAGTGCCAAGGATAGATTGTGTTTCAAAG | |
| | AATGAAGATATACCAAATGTAGCAGTTTATCCTCATAA | |
| | TGGAATGATAGACTTAAAATATTTCCCATATTATGGGA | |
| | AAAAACTGCATGTTGGGTATCTACAGCCATTGGTTGCTG | |
| | TTCAGGTCAGCTTTGCTCCTAACAACACTGGGAAAGAA | |
| | GTAACAGTTGAGTGCAAGATTGATGGATCAGCCAACCT | |
| | AAAAAGTCAGGATGATCGTGACAAGTTTTTGGGACGAG | |
| | TTATGTTCAAAATCACAGCACGTGCA | |
| BSG | GCTGCCGGCACAGTCTTCACTACCGTAGAAGACCTTGGC | 7 |
| | TCCAAGATACTCCTCACCTGCTCCTTGAATGACAGCGCC | |
| | ACAGAGGTCACAGGGCACCGCTGGCTGAAGGGGGGCGT | |
| | GGTGCTGAAGGAGGACGCGCTGCCCGGCCAGAAAACGG | |
| | AGTTCAAGGTGGACTCCGACGACCAGTGGGGAGAGTAC | |
| | TCCTGCGTCTTCCTCCCCGAGCCCATGGGCACGGCCAAC | |
| | ATCCAGCTCCACGGGCCTCCCAGAGTGAAGGCTGTGAA | |
| | GTCGTCAGAACACATCAACGAGGGGGAGACGGCCATGC | |
| | TGGTCTGCAAGTCAGAGTCCGTGCCACCTGTCACTGACT | |
| | GGGCCTGGTACAAGATCACTGACTCTGAGGACAAGGCC | |
| | CTCATGAACGGCTCCGAGAGCAGGTTCTTCGTGAGTTCC | |
| | TCGCAGGGCCGGTCAGAGCTACACATTGAGAACCTGAA | |
| | CATGGAGGCCGACCCCGGCCAGTACCGGTGCAACGGCA | |
| | CCAGCTCCAAGGGCTCCGACCAGGCCATCATCACGCTC | |
| | CGCGTGCGCAGCCACCTGGCCGCCCTCTGGCCCTTCCTG | |
| | GGCATCGTGGCTGAGGTGCTGGTGCTGGTCACCATCATC | |
| | TTCATCTACGAGAAGCGCCGGAAGCCCGAGGACGTCCT | |
| | GGATGATGACGACGCCGGCTCTGCACCCCTGAAGAGCA | |
| | GCGGGCAGCACCAGAATGACAAAGGCAAGAACGTCCGC | |
| | CAGAGGAACTCTTCC | |
| LAMP1 | GCAATGTTTATGGTGAAAAATGGCAACGGGACCGCGTG | 8 |
| | CATAATGGCCAACTTCTCTGCTGCCTTCTCAGTGAACTA | |
| | CGACACCAAGAGTGGCCCTAAGAACATGACCTTTGACC | |
| | TGCCATCAGATGCCACAGTGGTGCTCAACCGCAGCTCCT | |
| | GTGGAAAAGAGAACACTTCTGACCCCAGTCTCGTGATT | |
| | GCTTTTGGAAGAGGACATACACTCACTCTCAATTTCACG | |
| | AGAAATGCAACACGTTACAGCGTCCAGCTCATGAGTTT | |
| | TGTTTATAACTTGTCAGACACACACCTTTTCCCCAATGC | |
| | GAGCTCCAAAGAAATCAAGACTGTGGAATCTATAACTG | |
| | ACATCAGGGCAGATATAGATAAAAAATACAGATGTGTT | |
| | AGTGGCACCCAGGTCCACATGAACAACGTGACCGTAAC | |
| | GCTCCATGATGCCACCATCCAGGCGTACCTTTCCAACAG | |
| | CAGCTTCAGCCGGGGAGAGACACGCTGTGAACAAGACA | |
| | GGCCTTCCCCAACCACAGCGCCCCCTGCGCCACCCAGC | |
| | CCCTCGCCCTCACCCGTGCCCAAGAGCCCCTCTGTGGAC | |
| | AAGTACAACGTGAGCGGCACCAACGGGACCTGCCTGCT | |
| | GGCCAGCATGGGGCTGCAGCTGAACCTCACCTATGAGA | |
| | GGAAGGACAACACGACGGTGACAAGGCTTCTCAACATC | |
| | AACCCCAACAAGACCTCGGCCAGCGGGAGCTGCGGCGC | |
| | CCACCTGGTGACTCTGGAGCTGCACAGCGAGGGCACCA | |
| | CCGTCCTGCTCTTCCAGTTCGGGATGAATGCAAGTTCTA | |
| | GCCGGTTTTTCCTACAAGGAATCCAGTTGAATACAATTC | |
| | TTCCTGACGCCAGAGACCCTGCCTTTAAAGCTGCCAACG | |
| | GCTCCCTGCGAGCGCTGCAGGCCACAGTCGGCAATTCCT | |
| | ACAAGTGCAACGCGGAGGAGCACGTCCGTGTCACGAAG | |
| | GCGTTTTCAGTCAATATATTCAAAGTGTGGGTCCAGGCT | |
| | TTCAAGGTGGAAGGTGGCCAGTTTGGCTCTGTGGAGGAG | |
| | TGTCTGCTGGACGAGAACAGCATGCTGATCCCCATCGCT | |
| | GTGGGTGGTGCCCTGGCGGGGCTGGTCCTCATCGTCCTC | |
| | ATCGCCTACCTCGTCGGCAGGAAGAGGAGTCACGCAGG | |
| | CTACCAGACTATC | |
| LAMP2 | TTGGAACTTAATTTGACAGATTCAGAAAATGCCACTTGC | 9 |
| | CTTTATGCAAAATGGCAGATGAATTTCACAGTACGCTAT | |
| | GAAACTACAAATAAAACTTATAAAACTGTAACCATTTC | |
| | AGACCATGGCACTGTGACATATAATGGAAGCATTTGTG | |
| | GGGATGATCAGAATGGTCCCAAAATAGCAGTGCAGTTC | |
| | GGACCTGGCTTTTCCTGGATTGCGAATTTTACCAAGGCA | |
| | GCATCTACTTATTCAATTGACAGCGTCTCATTTTCCTAC | |
| | AACACTGGTGATAACACAACATTTCCTGATGCTGAAGA | |
| | TAAAGGAATTCTTACTGTTGATGAACTTTTGGCCATCAG | |
| | AATTCCATTGAATGACCTTTTTAGATGCAATAGTTTATC | |
| | AACTTTGGAAAAGAATGATGTTGTCCAACACTACTGGG | |
| | ATGTTCTTGTACAAGCTTTTGTCCAAAATGGCACAGTGA | |
| | GCACAAATGAGTTCCTGTGTGATAAAGACAAAACTTCA | |
| | ACAGTGGCACCCACCATACACACCACTGTGCCATCTCC | |
| | TACTACAACACCTACTCCAAAGGAAAAACCAGAAGCTG | |
| | GAACCTATTCAGTTAATAATGGCAATGATACTTGTCTGC | |
| | TGGCTACCATGGGGCTGCAGCTGAACATCACTCAGGAT | |
| | AAGGTTGCTTCAGTTATTAACATCAACCCCAATACAAC | |
| | TCACTCCACAGGCAGCTGCCGTTCTCACACTGCTCTACT | |
| | TAGACTCAATAGCAGCACCATTAAGTATCTAGACTTTG | |
| | TCTTTGCTGTGAAAAATGAAAACCGATTTTATCTGAAGG | |
| | AAGTGAACATCAGCATGTATTTGGTTAATGGCTCCGTTT | |
| | TCAGCATTGCAAATAACAATCTCAGCTACTGGGATGCC | |
| | CCCCTGGGAAGTTCTTATATGTGCAACAAAGAGCAGAC | |
| | TGTTTCAGTGTCTGGAGCATTTCAGATAAATACCTTTGA | |
| | TCTAAGGGTTCAGCCTTTCAATGTGACACAAGGAAAGT | |
| | ATTCTACAGCTGAAGAATGTTCTGCTGACTCTGACCTCA | |
| | ACTTTCTTATTGCTGTTGCAGTGGGTGTGGCCTTGGGCT | |
| | TCCTTATAATTGTTGTCTTTATCTCTTATATGATTGGAA | |
| | GAAGGAAAAGTCGTACTGGTTATCAGTCTGTG | |
| trastuzumab 3cFv | GAAGTGCAGCTGGTGGAAAGCGGCGGGGGCCTGGTGCAG | 10 |
| | CCCGGGGGAAGCCTGCGGCTGAGTTGCGCCGCTTCTGGC | |
| | TTCAACATCAAAGATACCTACATCCACTGGGTGAGACA | |
| | GGCCCCTGGCAAAGGCCTGGAGTGGGGGCCAGAATCT | |
| | ACCCCACAAATGGCTACACCAGGTATGCCGATAGCGTC | |
| | AAGGGCAGATTTACCATCAGCGCCGACACTTCAAAGAA | |
| | CACCGCCTATCTGCAGATGAACTCCCTGCGCGCCGAAG | |
| | ACACCGCCGTGTACTACTGTTCTCGGTGGGGCGGCGATG | |
| | GCTTCTATGCCATGGACTACTGGGGCCAGGGCACACTGG | |
| | TGACCGTGTCTAGCGGGGGAAGCTCCAGGTCCAGCTCTT | |
| | CCGGCGGCGGCGGCTCCGGCGGAGGAGGCGACATCCAG | |
| | ATGACCCAGTCACCAAGTTCTCTGAGCGCTTCCGTGGGC | |
| | GATCGCGTGACCATCACCTGCCGGGCTTCACAGGACGTG | |
| | AACACCGCCGTGGCCTGGTACCAGCAGAAACCTGGCAA | |
| | AGCCCCCAAACTCCTGATCTACTCCGCCAGTTTCCTGTA | |
| | TTCTGGCGTTCCTAGTAGATTTAGTGGAAGCAGGAGCGG | |
| | GACTGATTTCACTCTGACCATTAGCTCTCTGCAGCCTGA | |
| | GGACTTTGCCACATATTATTGCCAGCAGCACTACACCA | |
| | CCCCCCCAACTTTCGGCCAGGGCACCAAGGTCGAGATC | |
| | AAA | |

### INDUSTRIAL APPLICABILITY

The present invention relates to engineered cell-derived vesicles (CDVs) that may be used as a drug delivery system, and was completed by discovering four types of anchor proteins that match the intrinsic characteristics of CDVs and may mediate the stable introduction of biologically active molecules. The anchor proteins are CDV-specific membrane proteins that are abundantly present, and it was confirmed that CDVs comprising the anchor proteins may be more stably loaded with biologically active molecules. For example, as a result of carrying out comparative experiments by using a fluorescent protein, it was confirmed that CDVs into which the anchor proteins are introduced were more effectively loaded with the fluorescent protein, compared to CDVs without the anchor proteins. It was also confirmed that, when a cancer cell-targeting antibody was loaded into the engineered CDVs of the present invention, the engineered CDVs exhibited an increased ability to target cancer cells and were more effectively absorbed into cancer cells. That is, the CDVs of the present invention are BioDrone engineered with the anchor proteins and may be stably loaded with various biologically active molecules and deliver same to a target of interest, and thus are expected to be used as a platform for the delivery of various drugs and treatment.

1. A cell-derived vesicle in which an anchor protein is overexpressed, wherein the anchor protein is at least one selected from the group consisting of Basigin, ATP1B3, LAMP1, and LAMP2.

2. The cell-derived vesicle of claim 1, wherein the anchor protein is inserted into the membrane of the cell-derived vesicle.

3. The cell-derived vesicle of claim 1, wherein the cell-derived vesicle is derived from a cell in which the anchor protein is overexpressed.

4. The cell-derived vesicle of claim 3, wherein the cell-derived vesicle is obtained by extruding the cell.

5. The cell-derived vesicle of claim 3, wherein the cell is at least one selected from the group consisting of stem cells, immune cells, blood cells, embryonic cells, adipocytes, and embryonic kidney cells.

6. The cell-derived vesicle of claim 1, wherein the anchor protein is present at a higher level in the cell-derived vesicle than a cell from which the cell-derived vesicle is derived or an exosome produced from the cell.

7. The cell-derived vesicle of claim 1, wherein the anchor protein binds to a biologically active molecule.

8. The cell-derived vesicle of claim 7, wherein the biologically active molecule is located outside or inside the membrane of the cell-derived vesicle.

9. The cell-derived vesicle of claim 7, wherein the biologically active molecule is at least one selected from the group consisting of peptides, proteins, glycoproteins, nucleic acids, carbohydrates, lipids, glycolipids, compounds, natural products, viruses, semi-synthetic drugs, quantum dots, fluorochromes, and toxins.

10. The cell-derived vesicle of claim 9, wherein the protein is at least one selected from the group consisting of an antibody, an antibody fragment, a growth factor, an enzyme, a nuclease, a transcription factor, an antigenic peptide, a hormone, a transport protein, an immunoglobulin, a structural protein, a motor protein, a signaling protein, a linker protein, a viral protein, a natural protein, a recombinant protein, a protein complex, a fluorescent protein, a therapeutic protein, a chemically modified protein, and prions.

11. The cell-derived vesicle of claim 10, wherein the antibody is at least one selected from the group consisting of a full-length antibody, Fab, Fab′, F(ab′)₂, scFv, (scFv)₂, scFv-Fc, a minibody, a diabody, and a nanobody.

12. The cell-derived vesicle of claim 7, wherein the biologically active molecule is a targeting ligand, and the cell-derived vesicle binds to a cell expressing a target of the targeting ligand.

13-18. (canceled)

19. A drug delivery method comprising administering a cell-derived vesicle overexpressing at least one anchor protein selected from the group consisting of Basigin, ATP1B3, LAMP1, and LAMP2 to a subject in need thereof, wherein the cell-derived vesicle is loaded with a drug.

20. The drug delivery method of claim 19, wherein the drug is at least one selected from the group consisting of an antibody or fragment thereof, a therapeutic protein, and a therapeutic peptide.

21. The drug delivery method of claim 19, wherein the drug is bound to the anchor protein of the cell-derived vesicle; or loaded into the inside or membrane of the cell-derived vesicle.

22. The drug delivery method of claim 19, wherein the cell-derived vesicle further comprises a targeting ligand, and the targeting ligand is bound to the anchor protein and positioned outside the membrane of the cell-derived vesicle.

23. The drug delivery method of claim 22, wherein the cell-derived vesicle binds to a cell expressing a target of the targeting ligand.

24. A method for preventing or treating cancer comprising administering a cell-derived vesicle overexpressing at least one anchor protein selected from the group consisting of Basigin, ATP1B3, LAMP1, and LAMP2 to a subject in need thereof, wherein the cell-derived vesicle is loaded with an anticancer agent.

25. The method for preventing or treating cancer of claim 24, wherein the anticancer agent is bound to an anchor protein.

26-31. (canceled)
